(19) United States
(12) Patent Application Publication
(10) Pub. No.: US 2026/0144928 A1
(43) Pub. Date: May 28, 2026

(54) INSULIN PATCH PUMP

(71) Applicant: Tandem Diabetes Care Switzerland Sàrl, St-Sulpice (CH)

(72) Inventors: Nathanael Sigg, Oulens-sous-Echallens (CH); Zbynek Struzka, St-Sulpice (CH); Laurent Mosimann, St-Sulpice (CH); Foucauld Tabourin, St-Sulpice (CH)

(21) Appl. No.: 19/400,657

(22) Filed: Nov. 25, 2025

Related U.S. Application Data
(60) Provisional application No. 63/740,131, filed on Dec. 30, 2024.
(60) Provisional application No. 63/733,632, filed on Dec. 13, 2024.
(60) Provisional application No. 63/733,599, filed on Dec. 13, 2024.
(60) Provisional application No. 63/733,593, filed on Dec. 13, 2024.
(60) Provisional application No. 63/724,723, filed on Nov. 25, 2024.

(51) Int. Cl. A61M 5/142; A61M 5/145; A61M 5/172

(52) U.S. Cl. CPC A61M 5/14248; A61M 5/14566; A61M 5/172; A61M 2205/18; A61M 2205/582

(57) ABSTRACT

Provided herein are systems and methods for delivering medication, such as insulin, that are user-friendly, environmentally-friendly, lower cost, discreet, less prone to errors, and/or that deliver precise, repeatable doses of medication, as well as accessories for applying and managing the same. In embodiments, the system includes a wearable insulin pump having a patch-style form factor for adhesion to a user's body surface.

### PRIORITY CLAIM

The present application claims the benefit of U.S. Provisional Application No. 63/724,723 filed November 25, 2024, U.S. Provisional Application No. 63/733,593 filed December 13, 2024, U.S. Provisional Application No. 63/733,599 filed December 13, 2024, U.S. Provisional Application No.63/733,632 filed December 13, 2024, and U.S. Provisional Application No. 63/740,131 filed December 30, 2024, each of which is hereby incorporated by reference herein in its entirety.

### TECHNICAL FIELD

The present disclosure relates generally to systems and methods for delivering medication such as insulin to a user, for example, wearable insulin pumps having a patch-style form factor for adhesion to a user’s body surface.

### BACKGROUND

Wearable insulin pumps are known for providing a Type I Diabetes Mellitus patient with small doses of short acting insulin continuously (basal rate). The devices also can be used to deliver variable amounts of insulin when a meal is consumed (bolus). The basal insulin rates are usually programmed in a pump by a physician, and one or multiple basal settings may be programmed in the pump based on the patient's needs. The patient may program the amount of insulin for a mealtime bolus directly on the pump. Most pumps also include bolus calculators to help the patient determine the amount of insulin the patient may need at mealtime based on the patient's glucose levels and the amount of carbohydrates the patient may consume. The objective is to control the patient's blood glucose level within a desired range. Some such insulin pumps are coupled to an adhesive patch that permits the pump to be directly adhered to a user's body surface, for example the abdomen, and are referred to as “patch pumps.” In addition, some previously known systems were configured to interface wirelessly with a continuous glucose monitor, which typically also may be disposed on a patch designed to be adhered to the user's body. Other previously known systems employ still further modules designed to monitor user activity and report that activity to a controller associated with the patch pump to titrate the insulin delivery in accordance with the user's activity level.

U.S. Pat. No. 11,806,502, the entire contents of which are incorporated herein by reference, assigned to the assignee of the instant application, describes a self-contained patch pump having a motor-actuated syringe together with a microdosing pump chamber.

U.S. Pat. No. 11,813,428, the entire contents of which are incorporated herein by reference, assigned to the assignee of the instant application, describes a drug delivery device comprising a pumping system and a liquid reservoir fluidly connected to a delivery system outlet. The liquid reservoir has an elastic plunger sealingly slidable within a container wall of the liquid reservoir for expelling liquid out of the reservoir.

U.S. Pat. No. 11,529,460, the entire contents of which are incorporated herein by reference, assigned to the assignee of the instant application, describes systems and methods for delivering medication such as insulin that are user-friendly, environmentally-friendly, lower cost, discreet, less prone to errors, and/or that deliver precise, repeatable doses of medication.

### SUMMARY

Provided herein are systems and methods for delivering medication, such as insulin, that are user-friendly, environmentally-friendly, lower cost, discreet, less prone to errors, and/or that deliver precise, repeatable doses of medication, as well as accessories for applying and managing the same. In embodiments, the system includes a wearable insulin pump having a patch-style form factor for adhesion to a user's body surface.

In an embodiment, a medication infusion device includes a patch pump configured to be removably adhered to a wearer’s skin for delivering doses of medication to the wearer through a transcutaneous portion. The patch pump can include a pump motor disposed within the pump housing configured to pump the medication towards the transcutaneous portion. A vibration motor separate from the pump motor can be disposed within the pump housing. A controller can be operatively coupled to the pump motor and the vibration motor and configured to cause the vibration motor to vibrate to provide alerts to the wearer. A vibration holder can partially surround a portion of the vibration motor, the vibration holder configured to absorb the vibrations of the vibration motor to transmit the vibrations to the wearer external of the pump housing while preventing the vibrations from affecting other components internal to the pump housing.

In an embodiment, a medication infusion device includes a patch pump configured to be removably adhered to a wearer’s skin for delivering doses of medication to the wearer through a transcutaneous portion. The patch pump can include a pump housing including a main pump housing and a pump housing back configured to be adhered to each other. A pump motor can be disposed within the pump housing. A plunger can be connected to the pump motor by a pusher such that operation of the pump motor causes the pusher to move the plunger to deliver the medication to the wearer. The pusher can include a curved rod. A bender can be disposed between the curved rod and the housing to absorb forces imparted by the curved rod during pumping operations that would otherwise be imparted to the pump housing to prevent the pump housing back from separating from the main pump housing due at least in part to those forces.

In an embodiment, a medication infusion device can include a pump housing including a cartridge recess configured to receive a medication cartridge. A pump motor can be disposed within the pump housing that is configured to operate a pushing system to cause a medication in the medication cartridge to be delivered to the user. A sealing element can be disposed within the cartridge recess that is configured to provide a seal with the medication cartridge and the pump housing to prevent fluid from traveling from a wet zone region in the pump housing that includes the cartridge recess into a protected area of the pump housing that includes the pushing system.

In an embodiment, a medication infusion device comprises a patch pump configured to be removably adhered to a wearer’s skin for delivering doses of medication from a container through a transcutaneous portion that includes a pushing mechanism configured to be coupled to a plunger of the container. The pushing mechanism can include a screw and a nut configured to move along the screw in a first direction to advance a pusher to move the plunger. A pump motor can be coupled to the pushing mechanism and configured to move the pusher towards the plunger of the container by advancing the nut along the screw in the first direction such that medication is moved out of the container. The pump motor can be further configured to rewind the nut along the screw in a second direction opposite of the first direction when the container is empty of medication. A spring can provide a preloaded force onto the screw that counteracts forces imparted onto the screw by the pushing mechanism when the nut is rewound along the screw in the second direction.

In an embodiment, a medication infusion device comprises a patch pump configured to be removably adhered to a wearer’s skin for delivering doses of medication from a container through a transcutaneous portion that includes a pushing mechanism configured to be coupled to a plunger of the container. The pushing mechanism can include a screw and a nut configured to move along the screw in a first direction to advance a pusher to move the plunger. A pump motor can be coupled to the pushing mechanism and configured to move the pusher towards the plunger of the container by advancing the nut along the screw in the first direction such that medication is moved out of the container. The pump motor can be further configured to rewind the nut along the screw in a second direction opposite of the first direction when the container is empty of medication. The device can further include one or more contacting pins and a contacting blade affixed to the pushing mechanism that is configured to contact the one or more contacting pins when the pushing mechanism is fully rewound to send an electrical signal to a processor that the pushing mechanism is fully rewound. An electrical circuit formed when the contacting blade contacts the one or more contacting pins is configured to discharge any remaining accumulated charge.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is an exemplary medication infusion system according to the disclosure.

FIG. 2 is a diagram showing exemplary attachment zones for the patch pump of FIG. 1 and an external sensor.

FIGS. 3A-3B depict an exemplary patch pump according to the disclosure.

FIG. 4A-4B depicts an exploded view and a partial assembled view of the patch pump of FIGS. 3A-3B.

FIGS. 5A-5G depict portions of an exemplary patch pump according to the disclosure.

FIGS. 6A-6B depict the vibration holder of the patch pump of FIGS. 5A-5G.

FIGS. 7A-7E depict portions of an exemplary patch pump according to the disclosure.

FIGS. 8A-8B depict the inner bender of the patch pump of FIGS. 7A-7E.

FIGS. 8C-8D depicts the outer bender of the patch pump of FIGS. 7A-7E.

FIG. 9 depicts a cross-section plan view of the patch pump of wet and dry zones within the patch pump of FIGS. 3A-3B.

FIG. 10A-10B depict views of an exemplary sealing element for a patch pump according to the disclosure.

FIGS. 11A-11C depict portions of an exemplary patch pump according to the disclosure incorporating the seal of FIGS. 10A-10B.

FIGS. 12A-12B depict isolated partial views of the patch pump of FIGS. 11A-11C.

FIGS. 13A-13B depict an exemplary patch pump according to the disclosure.

FIG. 14 depicts a portion of an exemplary patch pump according to the disclosure.

FIGS. 15A-15D depict a portion of an exemplary patch pump according to the disclosure.

FIG. 16 depicts an end of stroke circuit according to the disclosure.

FIG. 17 depicts a portion of a patch pump system according to the disclosure.

FIG. 18 schematically depicts an aspect of a patch pump system according to the disclosure.

FIG. 19 schematically depicts an aspect of a patch pump system according to the disclosure.

FIG. 20 schematically depicts an aspect of a patch pump system according to the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

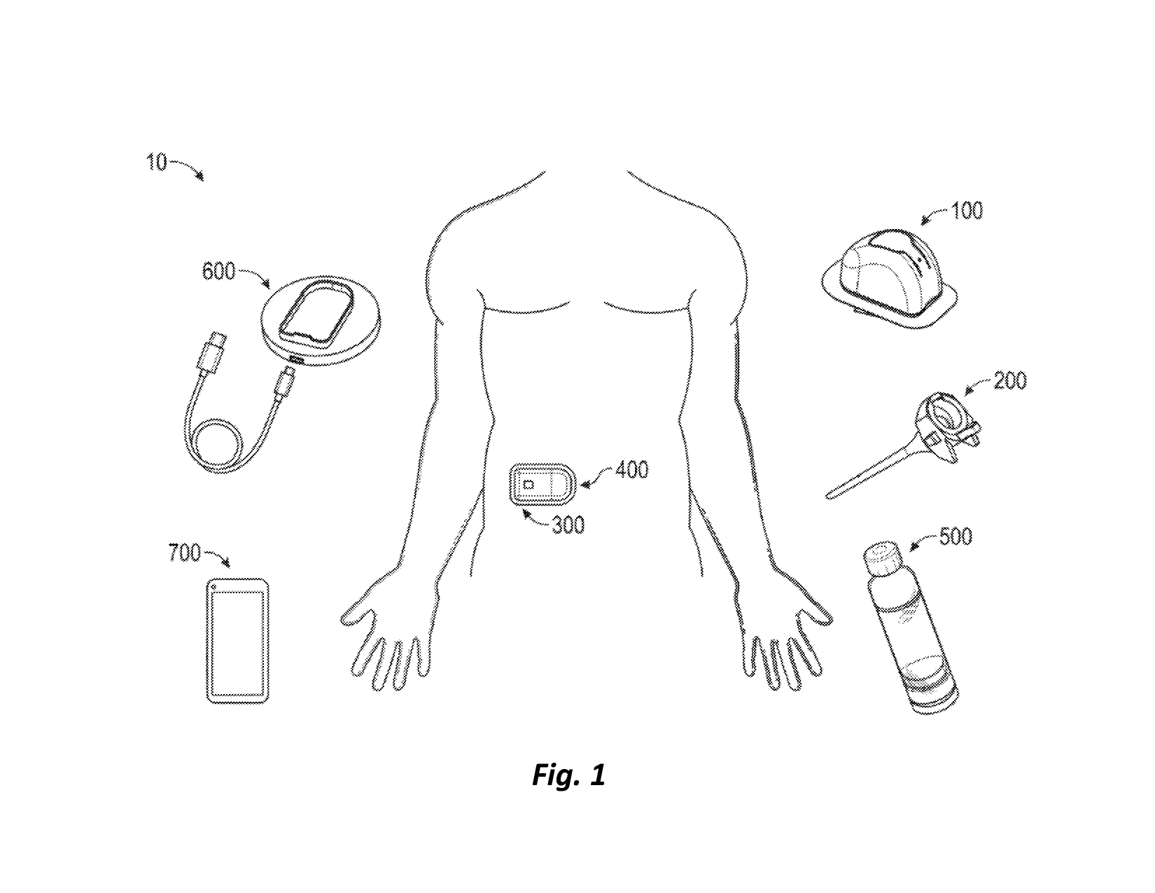

Referring to FIG. 1, an exemplary medication infusion system including a patch pump for delivering medication is described. In FIG. 1, components of the system are not depicted to scale on either a relative or absolute basis. Medication infusion system 10 can include applicator 100, cannula 200, pump 300, cap 400, cartridge 500, charging system 600 and/or software application 700. Preferably, applicator 100, cannula 200, cap 400, and cartridge 500 are disposable components that may be replaced approximately every 3-10 days and/or once the pre-filled cartridge is empty, while pump 300 is reusable and may last for an extended period of time, e.g., approximately 2-4 years. As such, pump 300 may be used with many different applicators, cannulas, caps, and pre-filled cartridges. Such a configuration is expected to promote sanitary use of the system, as the components exposed to the patient and the insulin are disposable, while reducing costs for components containing more expensive electronics, e.g., pump 300, charging system 600, and/or software application 700, which may be used repeatedly. In some embodiments, system 10 includes a second pump, such that the wearer may charge a battery of the second pump while using the first pump and vice versa. In this manner, the wearer will always have a pump that is charged and ready to be used once the cartridge of the pump in use is empty. Further, this system can be designed to reduce waste while reducing the number of times the wearer is required to insert a new cannula. Medication infusion system 10 may be used to apply cannula 200 and a pad to a wearer and to deliver medication through cannula 200 via a patch pump coupled to the pad. Further details regarding such a system can be found in U.S. Patent Publication No. 2022/0379014, which is hereby incorporated herein by reference in its entirety.

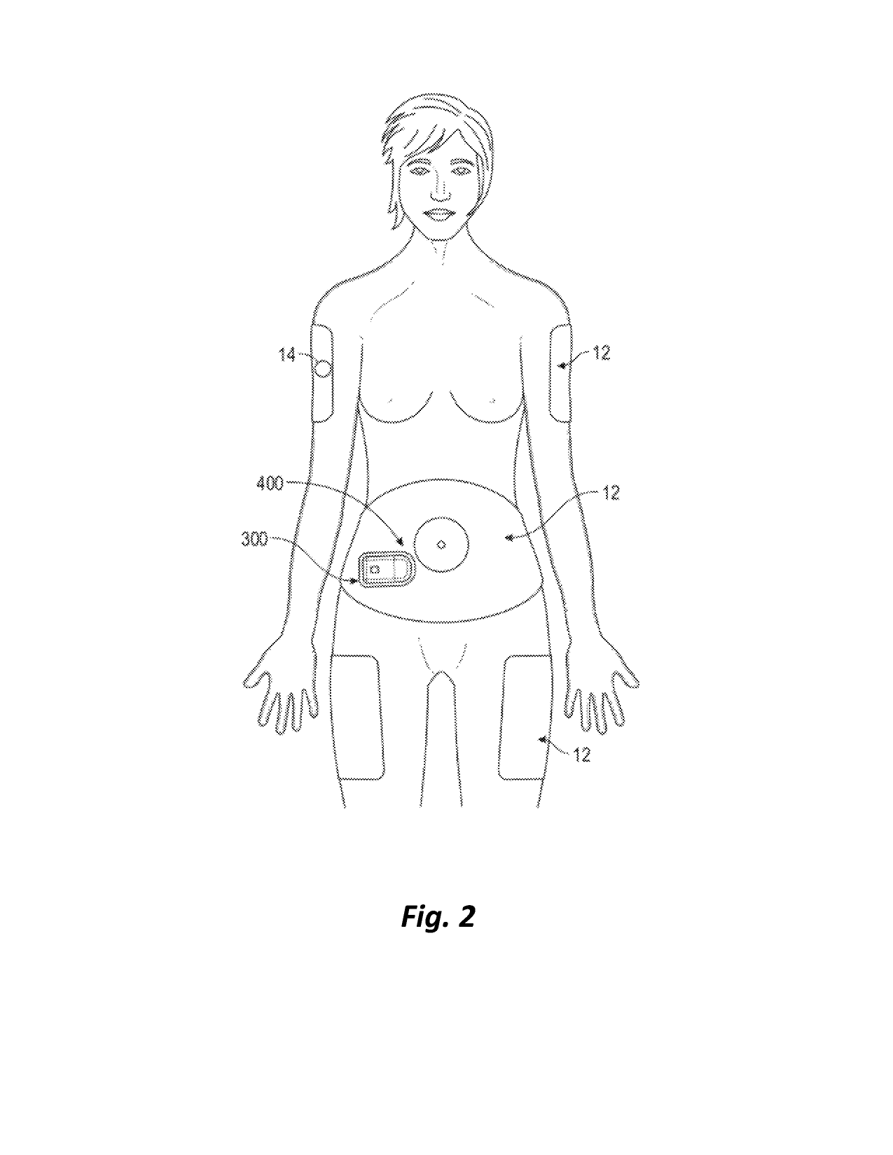

Referring now to FIG. 2, exemplary attachment zones for the patch pump and an optional external sensor, such as a continuous glucose monitoring sensor are illustrated. Attachment zones 12 illustrate several locations on the wearer's body where the applicator may attach the adhesive pad and insert the cannula and to which the patch pump is secured. For example, the patch pump may be secured to the upper arms, abdomen, or thighs of the wearer. As will also be understood by one of ordinary skill in the art, the patch pump may be secured to other locations on the wearer.

The patch pump also may be operatively coupled to an optional continuous glucose monitoring sensor, which may transmit data to a controller of the patch pump, which data may be used to adjust the time of insulin delivery or the amount of each dose. Preferably, the patch pump receives data from continuous glucose monitoring sensor 14, which is configured to be attached within attachment zones 12.

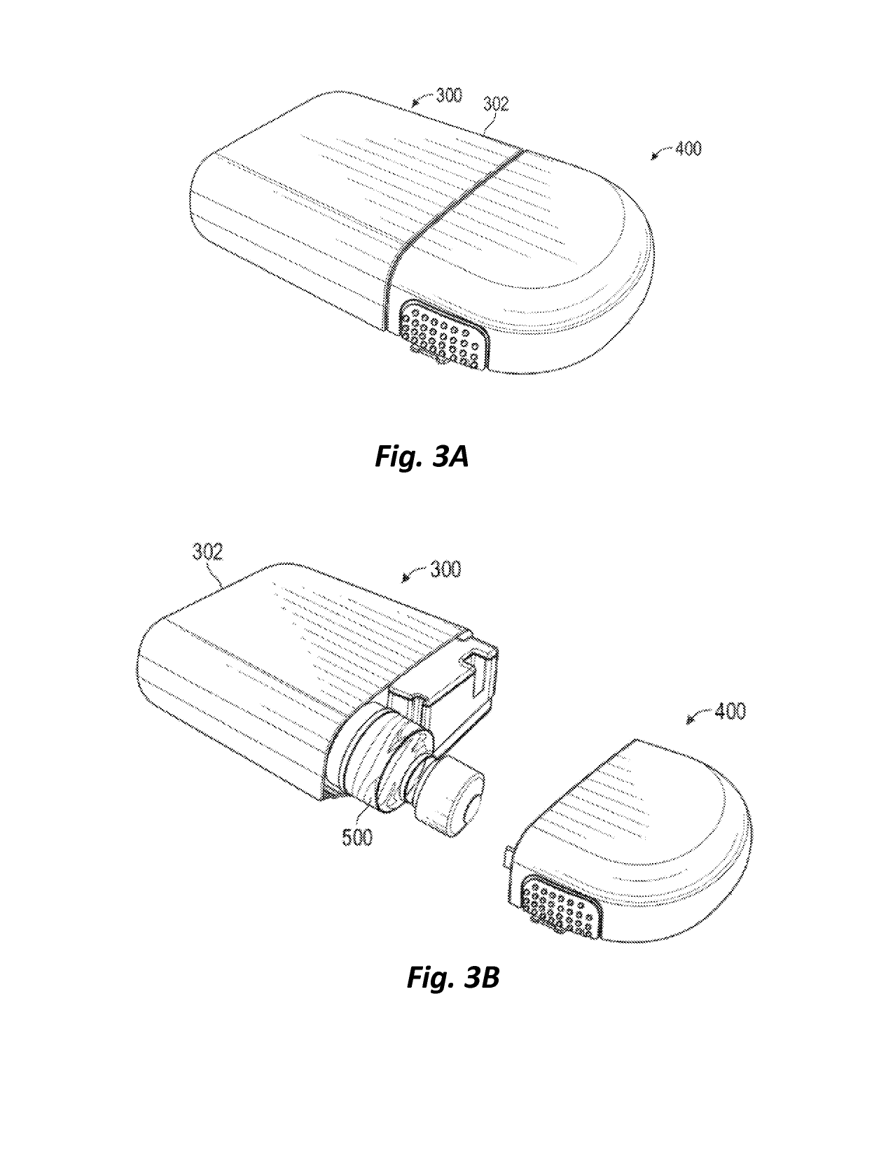

Referring now to FIGS. 3A and 3B, perspective views of an exemplary patch pump and pump-cap assembly according to the present disclosure are depicted. The patch pump is configured to attach to the adhesive pad secured to the wearer and to deliver doses of medication through the inserted cannula. The patch pump preferably includes a reusable pump, a disposable cap, a disposable pre-filled cartridge of medication, and a pad.

The patch pump may include pump 300 preferably designed to be used for an extended period of time (e.g., 2-4 years), and cap 400 preferably designed to be replaced after a much shorter period of time (e.g., 3-5 days). The patch pump also may include a pre-filled cartridge of medication, such as cartridge 500, which may be filled during manufacturing or by the wearer prior to inserting cartridge 500 into the pump. Cartridge 500 is configured to be inserted into the patch pump such that cartridge 500 is completely enclosed within the patch pump. For example, cartridge 500 may be inserted first into pump 300 such that a portion of cartridge 500 remains outside of pump housing 302. Cap 400 then may be coupled to cartridge 500 such that an inflow needle disposed within cap 400 pierces the cartridge cap of cartridge 500. While still maintaining inflow needle within cartridge 500, cap 400 then may be rotated relative to pump 300 to lock cap 400 to pump 300, thereby coupled the cap-pump assembly to the pad and the pump.

Pump 300 may include a motor disposed within pump housing 302, the motor configured to move a pusher coupled to the plunger of cartridge 500 such that insulin is advanced through an inflow needle of cap 400 and to a microdosing system designed to measure and deliver predetermined doses of medication. The same motor may simultaneously activate the plunger of the cartridge and the microdosing system, for example, via a gearbox. Doses of medication may be delivered to the user responsive to operation of a processor, in accordance with programming stored in memory associated with the processor or specifically when requested by the user, e.g., using a suitable wireless application on the user's smartphone. The processor may be configured to monitor one or more sensors and modify operation of pump 300 or alert the wearer based on information sensed by one or more sensors. Cap 400 is configured to receive medication from cartridge 500 and deliver predetermined doses of the medication through an outflow needle, into cannula 200, and to the wearer. Cap 400 preferably includes a microdosing system configured to measure and deliver the predetermined doses of medication.

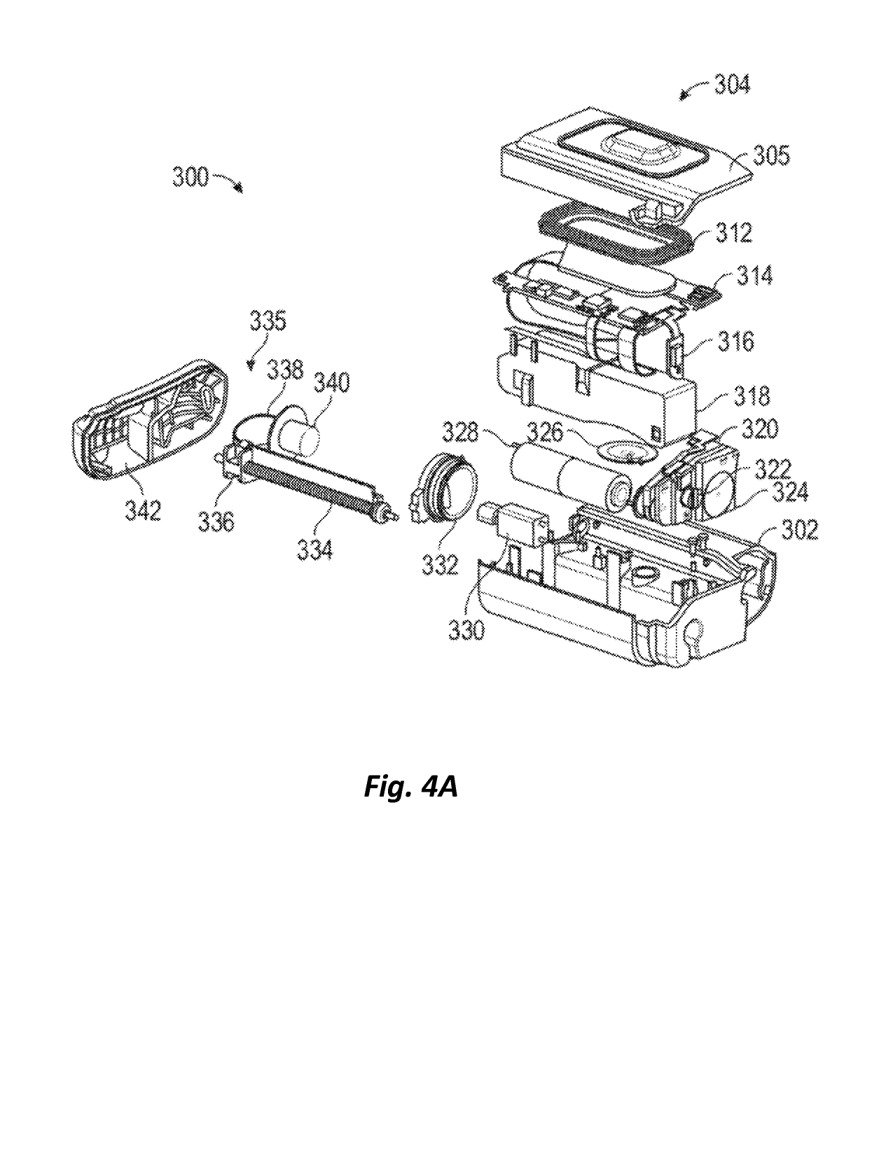

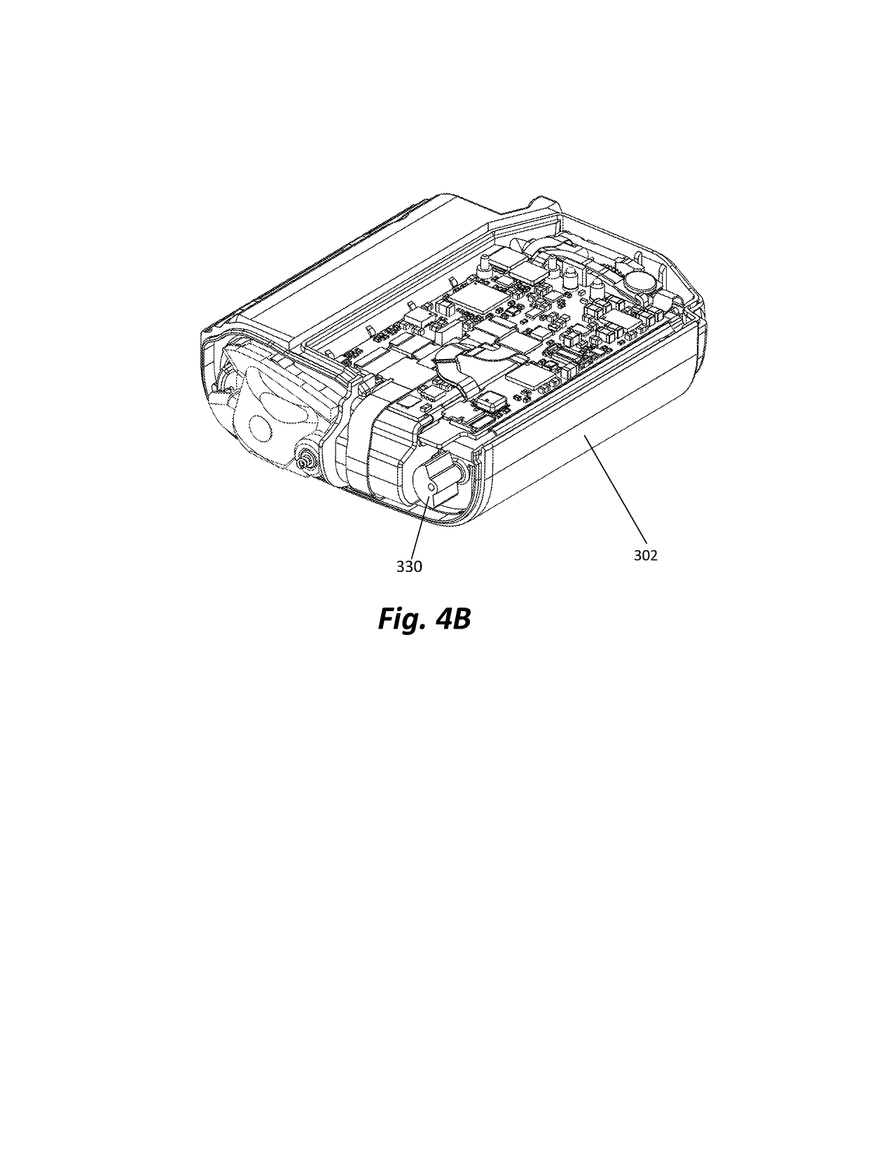

Referring now to FIG. 4A, internal components of an exemplary pump are described. For example, pump 300 may include within pump housing 302 and pump housing bottom 305 the following components: coil 312, circuit board 314, sensor 316, battery 318, sensor 320, mechanical coupling 322, gearbox 324, sound generator 326, pump motor 328, vibration motor 330, cartridge holder 332, and/or pusher 335 (which may include screw 334, nut 336, bendable rod 338, and/or cartridge contactor 340). Further details regarding these components can be found in U.S. Patent Publication No. 2023/0364332, which is hereby incorporated by reference herein in its entirety.

Pump 300 may include a housing having one or more separate pieces that are configured to couple together to enclose the internal components of the pump. Preferably, pump 300 includes a minimal number of parts such that the cost of the pump is reduced. For example, pump 300 may include pump housing 302 coupled to pump housing bottom 305, which may include photoplethysmography sensor frame 304. Pump housing 302 preferably has a cavity to receive a portion of a pre-filled cartridge. Pump housing 302 may further include pump housing back 342, which may be disposed on the end of pump housing 302 that does not lock with the cap. When coupled to the cap housing, the combined housings preferably fully enclose the cartridge.

### Reinforced Vibration Motor

The controller may be configured to cause the vibration motor 330 to vibrate to alert the wearer based on a parameter sensed by one or more sensors, for example, when the sensed parameter falls outside a predetermined threshold. Similarly, the controller further may be configured to determine that an error has occurred associated with operation of the patch pump based on the sensed parameter to cause the vibration motor to vibrate based on the determination of the error. For example, the sensor may be configured to sense a pressure within the cartridge, detect an occlusion within the dosing tube, monitor glucose levels of the wearer, sense at least one of the wearer's heart rate or other physiologic parameters, be an accelerometer sensing a parameter associated with the wearer's activity level and/or detect a temperature within the patch pump. The controller may be configured to cause the vibration motor to vibrate to provide an alert to the wearer based on any of these examples. Vibration motor can also vibrate for other reasons, such as, for example, to acknowledge a user action, to acknowledge the completion of a pump action, to give haptic feedback to selection of a touch-selectable button, etc.

As can be seen from FIG. 4B, the vibration motor 330 is situated within pump housing 302 in close proximity with a number of other internal components. It has been found that in the depicted configuration the vibrations from the vibration motor 330 could be transmitted to and cause issues with the functioning of the other internal components of the housing 302.

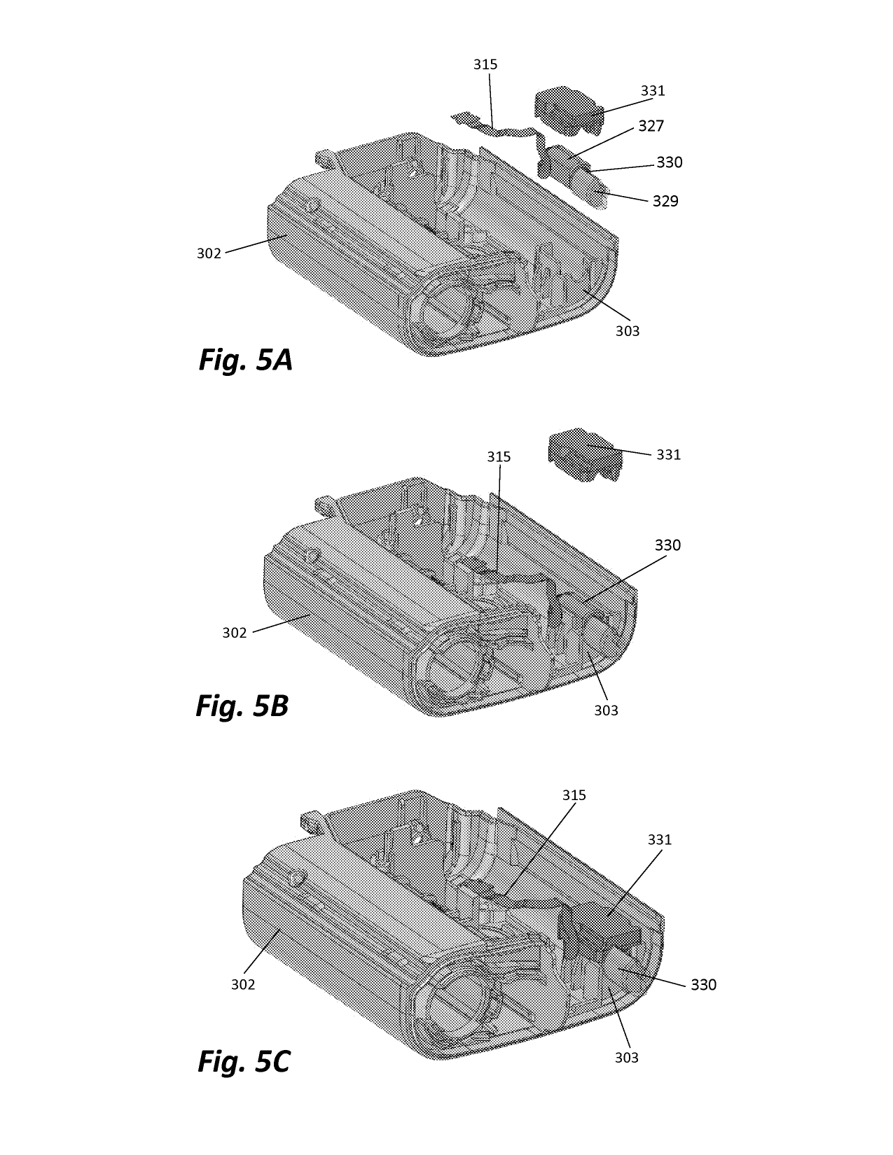

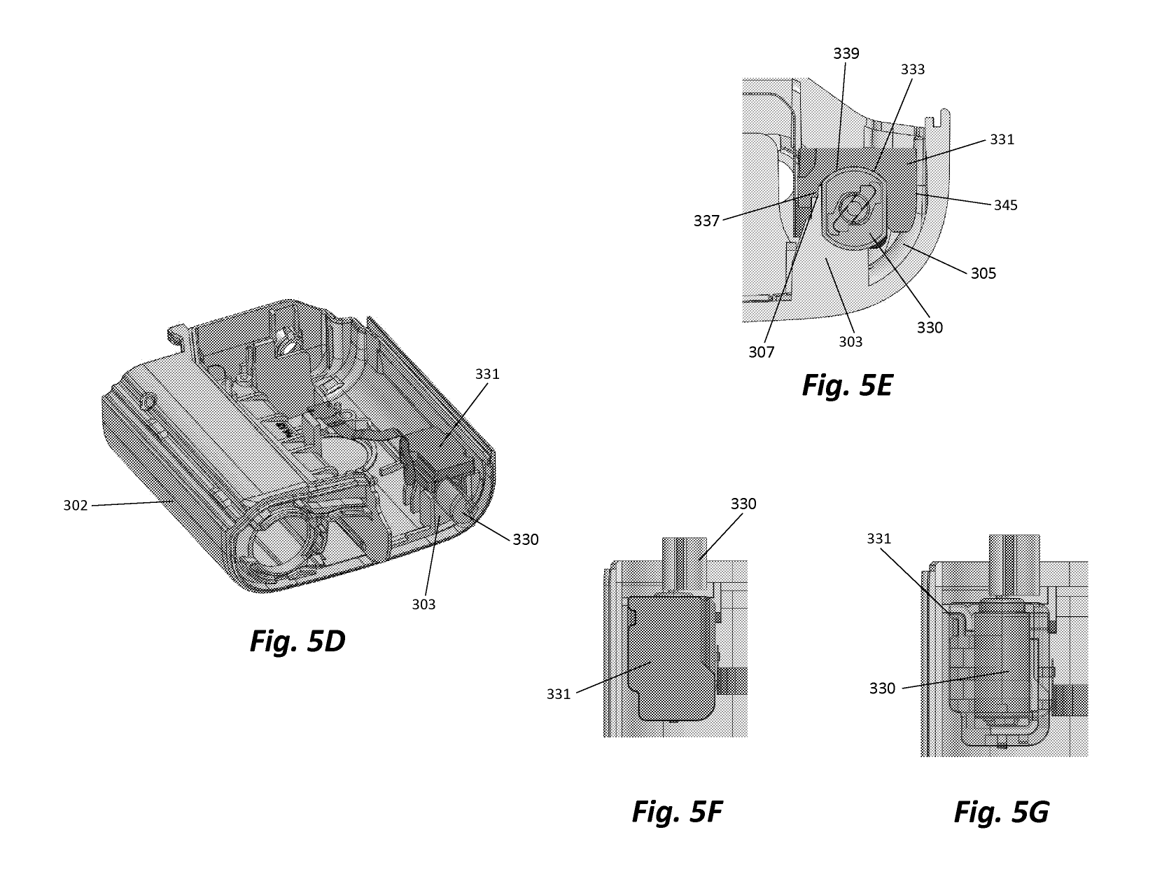

Referring now to FIGS. 5A-5G, a solution to this issue is depicted in which a vibration holder 331 is provided within the pump housing to absorb the vibrations from the vibration motor 330 and transmit the vibrations to the wearer without overly vibrating the other internal components of the pump. The vibration holder 331 ensures a tighter and more secure fit of the vibration motor 330 within the pump.

Vibration motor 330 can comprise a DC motor 327 with an eccentric rotating mass 329. When the DC motor 327 is energized, the eccentric mass 329 rotates to create the vibrations. The vibrations are then transmitted to the DC motor 327 then to the vibration holder 331 and from the vibration holder 331 through the pump housing 302 to the user.

Vibration motor 330 is received within motor nest 303 on the interior of pump housing 302. A flexible electrical connector 315 can electrically connect the vibration motor to the main circuit board of the pump to enable the pump controller to selectively cause the vibration motor to vibrate. Vibration holder 331 fits over and around the DC motor portion 327 of vibration motor 330 (because the eccentric mass 329 must be free to rotate). As can be seen in particular in FIG. 5E, an inner surface 333 of vibration holder conforms to a shape of the portion of vibration motor 331 and an outer surface 345 of vibration holder 331 is shaped to conform to an inner wall 305 of pump housing 302. In the depicted embodiment, vibration holder 331 extends around a first surface 339 of the portion of vibration motor and down along opposing sides of vibration component 330. A projection 337 of vibration holder 331 also nests on a ledge 307 of the motor nest 303 in which the vibration motor 330 is seated. This tight fit of vibration holder 331 between vibration motor 330 on motor nest 303 and pump housing 302 enables the vibration holder 331 to absorb the vibrations of the vibration motor 330 sufficient to translate the vibrations external of the pump housing 302 to alert the wearer without the vibrations affecting operation of the other internal components of the pump 300.

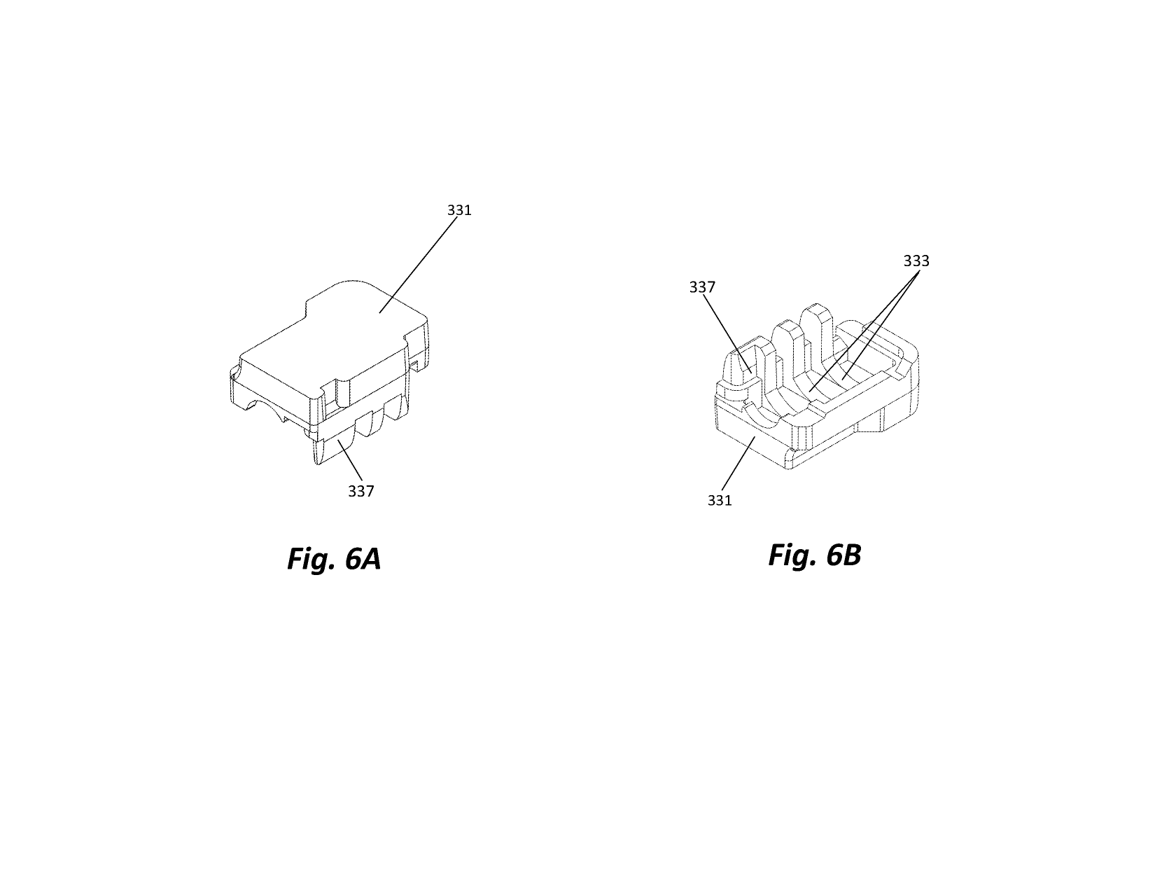

Vibration holder 331 can comprise a single, monolithic piece of material formed in the shape depicted in FIGS. 6A-6B. It should be noted that the shape depicted in these figures is exemplary only and that the vibration holder could take on many other shapes and forms based on a given pump and vibratory motor. Rather than any specific form factor, the critical design feature for vibration holder is to provide a contact point between the vibration motor and the housing to absorb and transmit the vibrations from the vibration motor to the pump housing. In embodiments, vibration holder 331 can be formed of a plastic material. Example materials can include, for example, polyamide, acrylonitrile butadiene styrene (ABS) and polypropylene. Vibration holder 331 can be bonded to the inner wall 305 of pump housing 302 by various means, including, for example, ultrasonic welding. Vibration holder 331 provides a tighter and more secure fit for vibration motor 330 within pump housing 302 such that vibrations from the vibration motor 330 can be transmitted to the wearer external of the pump housing 302 without any significant vibrations being absorbed by other internal components of the pump.

### Reinforced housing

Referring again to FIG. 4A, pushing system 335 is designed to push, responsive to movement from pump motor 328, on an end of the cartridge. Preferably, pushing system 335 pushes on a flexible plunger within the cartridge to move medication out of the cartridge during dosing. Pushing system 335 also may push the plunger of the cartridge to increase pressure in the cartridge without moving medication to the wearer, for example, during pump initialization. Pushing system 335 may include screw 334, nut 336, bendable or curved rod 338 and/or pusher 340. Screw 334 is coupled to pump motor 328, e.g., via gearbox 324. Screw 334 may be a worm screw. Responsive to rotational movement at pump motor 328, screw 334 rotates in a corresponding manner (e.g., at a geared ratio). Movement of screw 334 causes nut 336 to move along screw 334. Screw 334 may be a threaded screw and nut 336 may be a threaded nut that moves along the screw responsive to rotation of the screw. Curved rod 338 is coupled to nut 336 and moves as nut 336 moves. Curved rod 338 may curve in an approximately 180-degree angle to cause equal and opposite movements between nut 336 and pusher 340. Curved rod 338 enables operation of pusher in a smaller form factor. Pusher 340 is designed to contact the cartridge and move the plunger of the cartridge responsive to movement of pump motor 328. Pusher 340 may have a flange that contacts an outer surface of the plunger at a non-insulin-contacting end. Pusher 340 also may have an extension with a smaller diameter than the flange that extends into the inner part of the plunger. In this manner, pusher may have a top hat shape.

As can be seen in FIG. 4A, curved rod 338 is disposed within housing adjacent to the pump housing back 342. It has been found that curved rod 338 provides a force on the housing during pump operations (i.e., as nut 336 moves along screw 334 and force is imparted on curved rod 338 that causes the curved rod 338 to move the pusher 340 in the opposite direction, force is also transmitted from the outer curved surface of the curved rod onto the adjacent housing). In some circumstances, this force could cause the pump housing back 342 to break free and become disconnected from the main pump housing 302. This issue occurs most frequently in a situation where the pump 300 is dropped, with the force of the drop combined with the force of the curved rod 338 causing the pump housing back 342 to become disconnected.

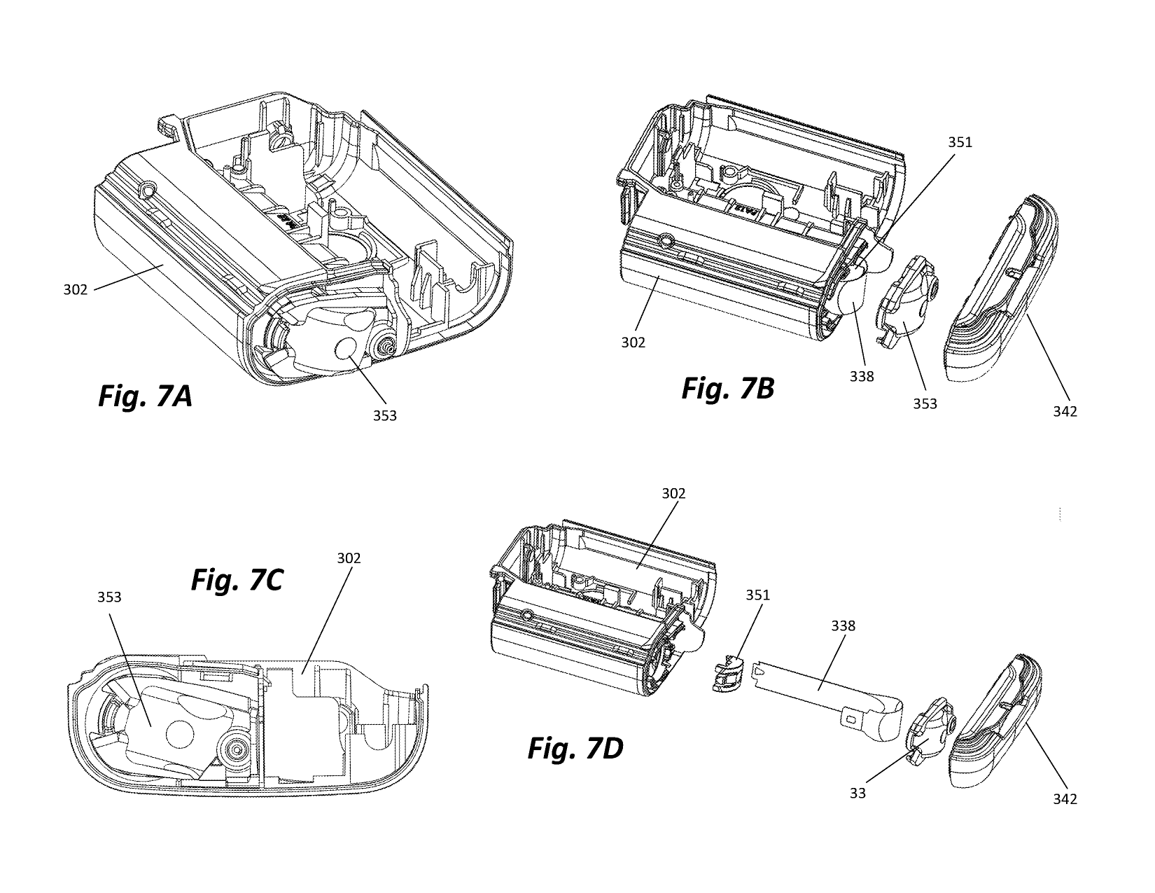

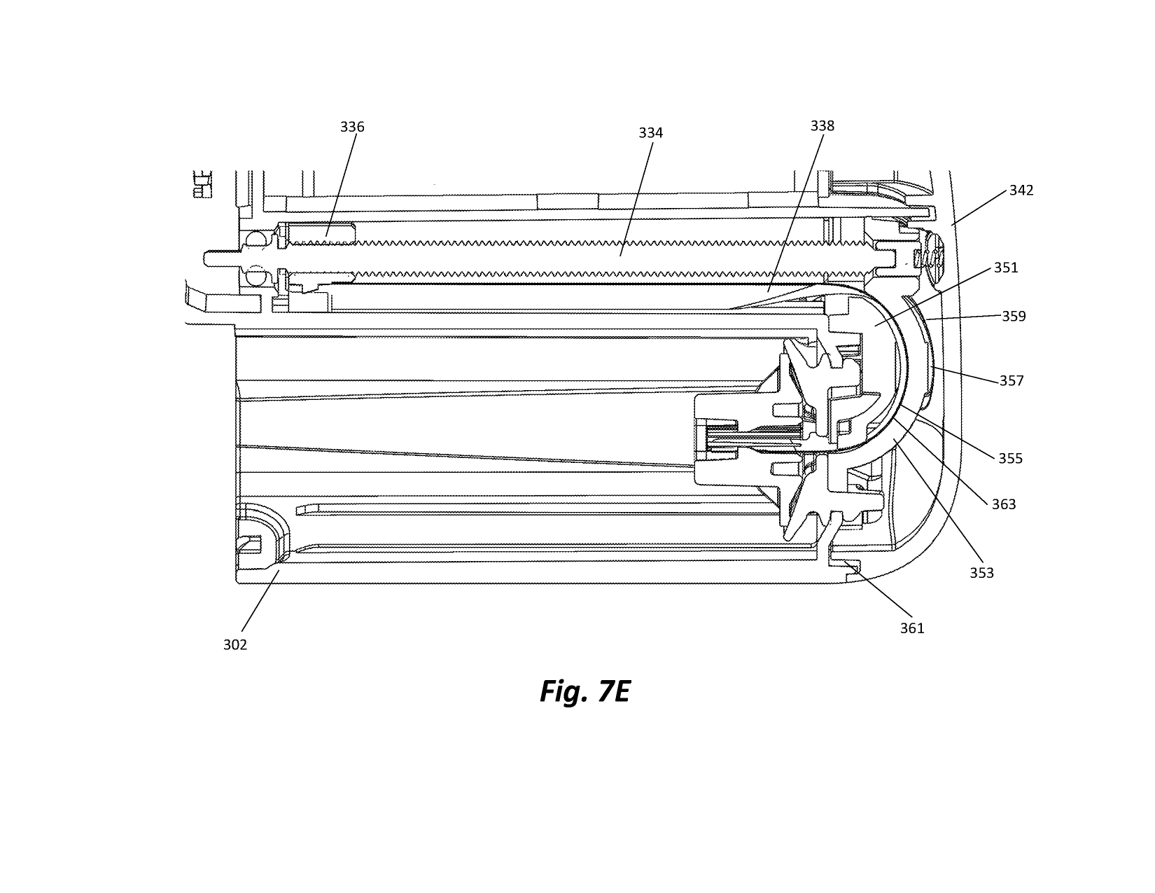

Referring now to FIGS. 7A-7E, aspects of a pump system according to the disclosure are depicted that address this issue. In particular, this embodiment includes a pair of cooperating features along inner and outer sides of the curved rod 338 that serve to absorb the force of the curved rod 338 previously directed onto the housing while maintaining the functionally of the curved rod 338.

For example, pump includes an inner bender 351 on an inside of the curved rod 338 and an outer bender 353 on an outside of the curved rod 338 between the curved rod 338 and the pump housing back 342. Outer bender 353 includes a curved inner surface 355 conforming to a shape of curved rod 338 and a curved outer surface 357 that interfaces with an inner surface 359 of pump housing back 342. Outer bender 353 is configured and positioned such that the forces provided by the curved rod 338 in the direction of the pump housing back 342 are absorbed by outer bender 353 and not transmitted to pump housing back 342. In particular, glue region 361 is an interface between main pump housing 302 and pump housing back 342 where an adhesive aids in holding the components together. The configuration and location of outer bender 353 is such that no recurring forces are imparted onto glue region 361 during pump operations, which would be the case were outer bender 353 not present. Inner bender 351 includes a curved outer surface 363 that conforms to the shape of curved rod 338 but does not contact the curved rod during pump operations. Inner bender 351 aids in ensuring proper rewinding of the pumping system 335 from an empty position to a full position when the cartridge is changed.

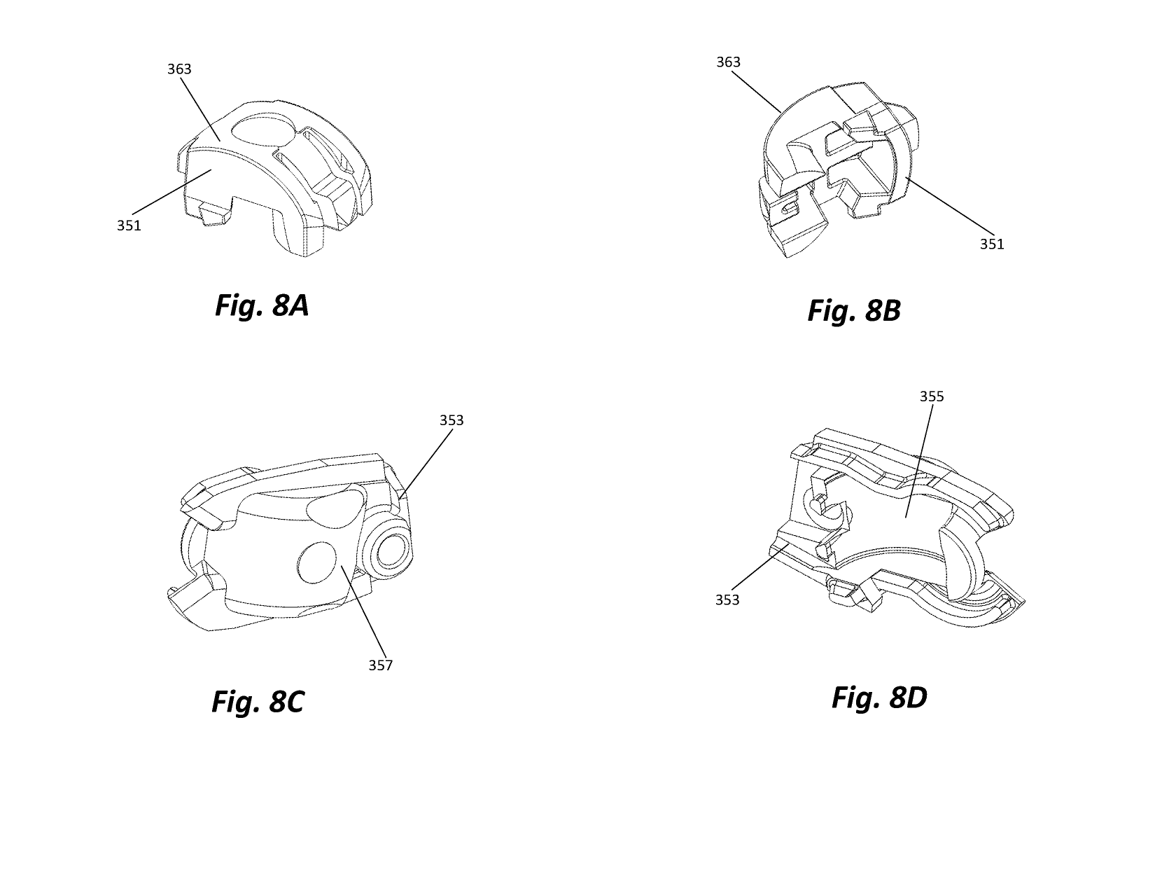

Inner bender 351 and/or outer bender 353 can each comprise a single, monolithic piece of material formed in the shapes depicted in FIGS. 8A-8D. In embodiments, these components can be formed of a plastic material. Example materials can include, for example, polyamide, polyamide, acrylonitrile butadiene styrene (ABS) and polypropylene. Inner bender 351 and/or outer bender 353 can be bonded to inner surfaces of pump housing by various means, including, for example, ultrasonic welding, with an adhesive, etc.

Outer bender 353 and inner bender 351 disclosed herein therefore reduce forces on the housing, while maintaining functionality of the curved rod 338, enabling a water-tight adhesive to be used to affix the pump housing back 342 onto the main pump housing 302 with a significantly reduced risk of the pump housing back 342 separating from the main pump housing 302 in certain circumstances such as when the device is dropped. As such, the pump disclosed herein has been shown to outperform pumps not having such components in drop testing.

### Enhanced Sealing

Pump housing 302 of pumps disclosed herein also may include a dry zone seal and/or one or more dry zone vents, which are configured to separate wet and dry zones of the pump such that the electrical components in the dry zone are isolated from the wet zone and do not contact any fluid and/or to permit humidity and gas to escape the housing such that pressure may equilibrate.

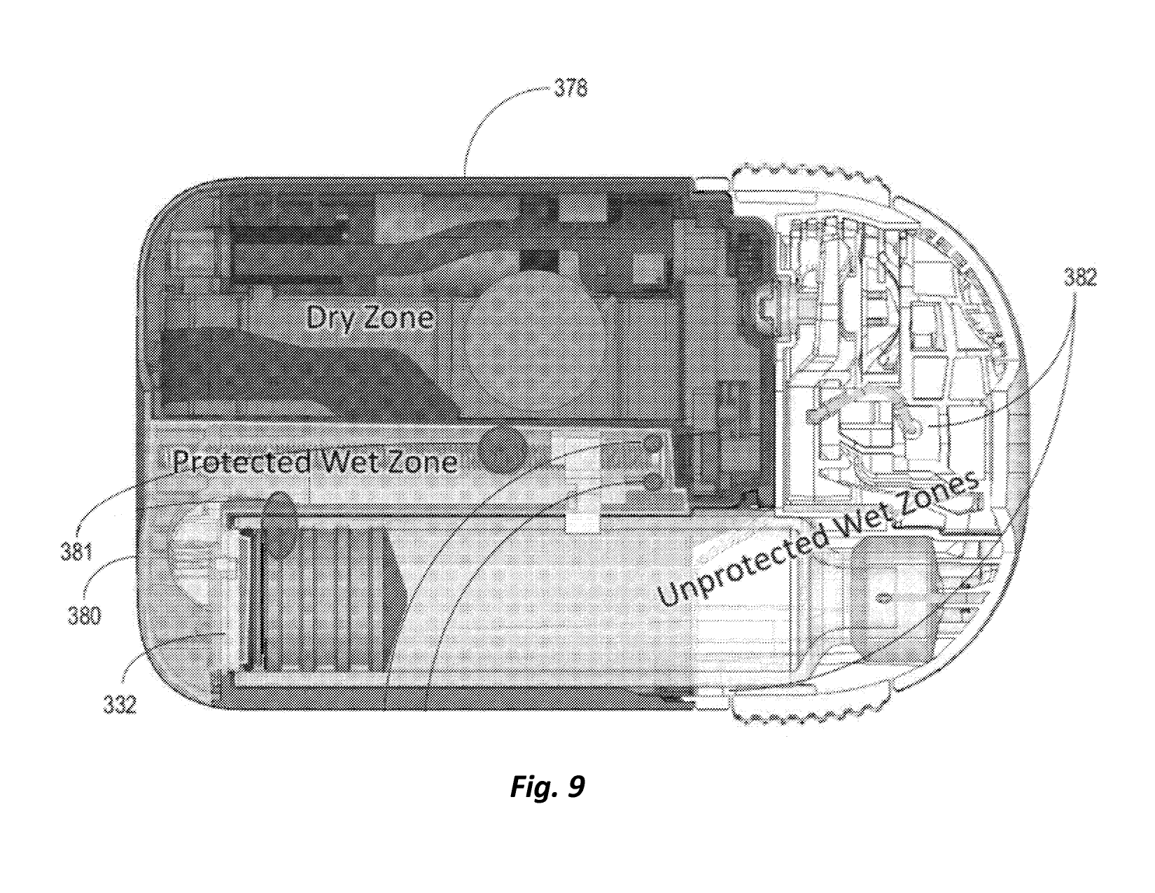

Referring to FIG. 9, an arrangement of wet and dry zones within the patch pump is described. The patch pump may include isolated wet and dry zones to protect the electrical components from contacting any leaked medication or other fluids. The patch pump may include dry zone 378 that is configured to house the circuit board, motor, vibration motor, sound generator, battery, coil, gearbox, and one or more sensors. Dry zone 378 may be encapsulated to exclude moisture from reaching the components within the zone. Dry zone 378 may be encased in plastic and/or sealed via welding or glueing. Dry zone 378 may be separated from protected wet zone 380 and unprotected wet zone 382 via one or more sealing members, for example, dry zone seal 379. Protected wet zone 380 may be configured to house the pushing system 335 and unprotected wet zone 382 may be configured to house the cartridge and the components of the cap including the needles and microdosing system. In addition, cartridge holder 332 may separate the protected wet zone from the dry zone and may include one or more O-rings to seal off the zones when a cartridge is disposed within the pump-cap assembly.

However, it has been found that even with the presence of such O-rings that fluid (e.g., insulin from within cartridge 500) can leak from the wet zone 382 into the protected wet zone 380 and migrate to a region near the end of the screw 334, inhibiting the functioning of screw 334 and thereby operation of the pushing system 335 and proper dispensing of insulin. Embodiments disclosed herein therefore provide a sealing element 385 that provides a more secure seal.

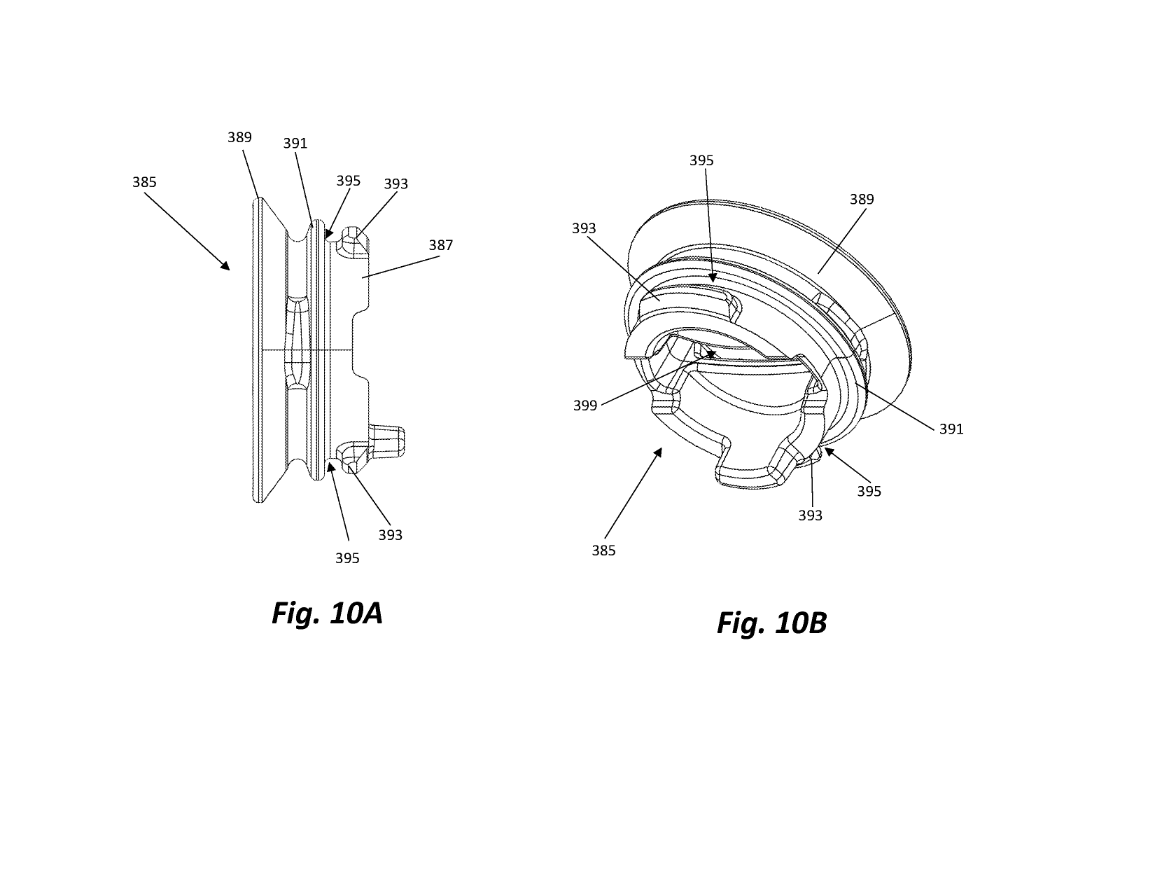

Sealing element 385 according to one example embodiment is depicted in FIGS. 10A-10B. Sealing element 385 includes a generally cylindrical body 387. A flange 389 can extend outwardly around cylindrical body 387 at a cartridge-facing end of sealing element 385. This flexible flange 389 enables sealing element 385 to accommodate a large tolerance in cartridge length as the flange 389 can bend downwardly to seal against slightly longer cartridges. Sealing element 385 can also include an annular rim 391 extending around cylindrical body 387. A pair of sealing projections 393 can extend outwardly from cylindrical body 387 adjacent the annular rim 391, creating a pair of sealing recesses 395 between annular rim 391 and sealing projections 393. This geometry is used to create fixation to the main plastic casing of the pump housing, as described in more detail below. A central opening 399 extends through cylindrical body 387 that enables the curved rod 338 of the pushing system 335 to pass through and interface with the cartridge 500. In embodiments, sealing element can comprise a single, monolithic piece of material formed in the shape depicted in FIGS. 10A-10B. In embodiments, sealing element 385 can comprise an elastomeric material, such as, for example, silicone.

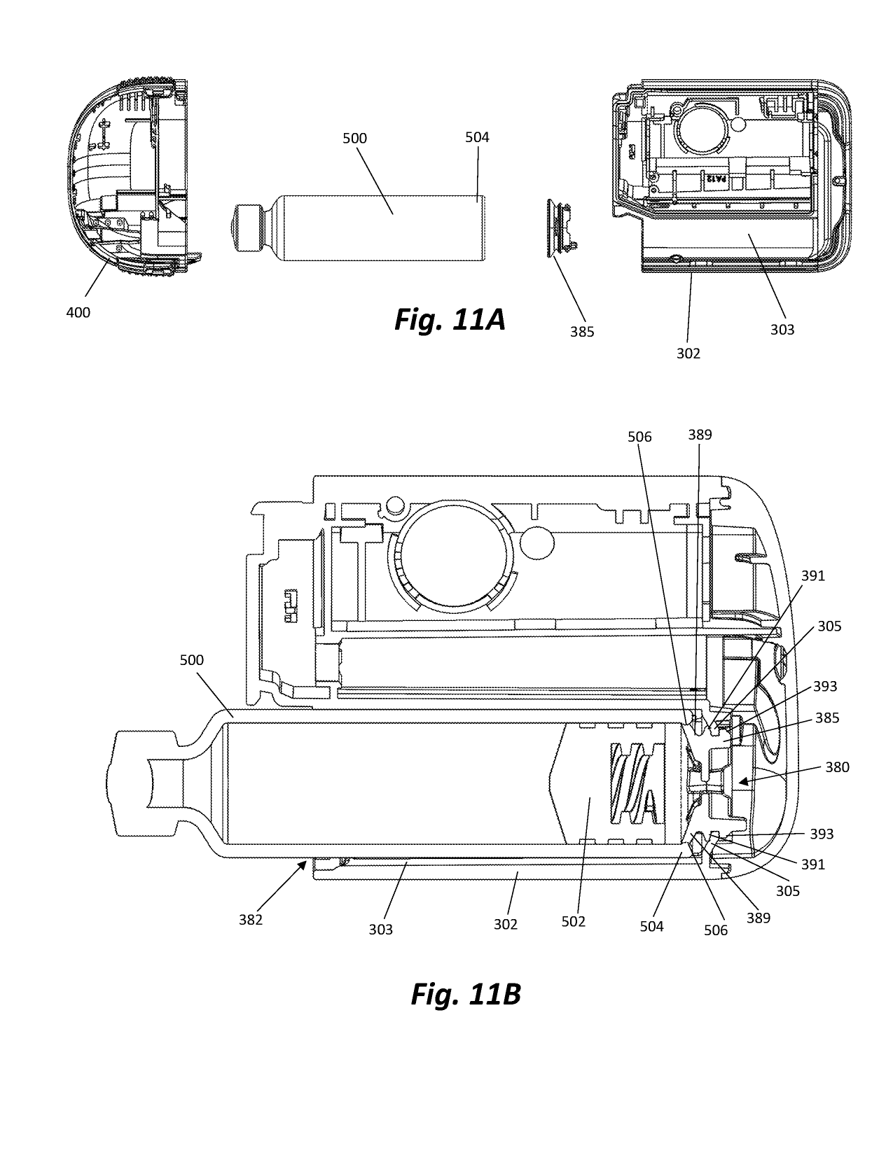

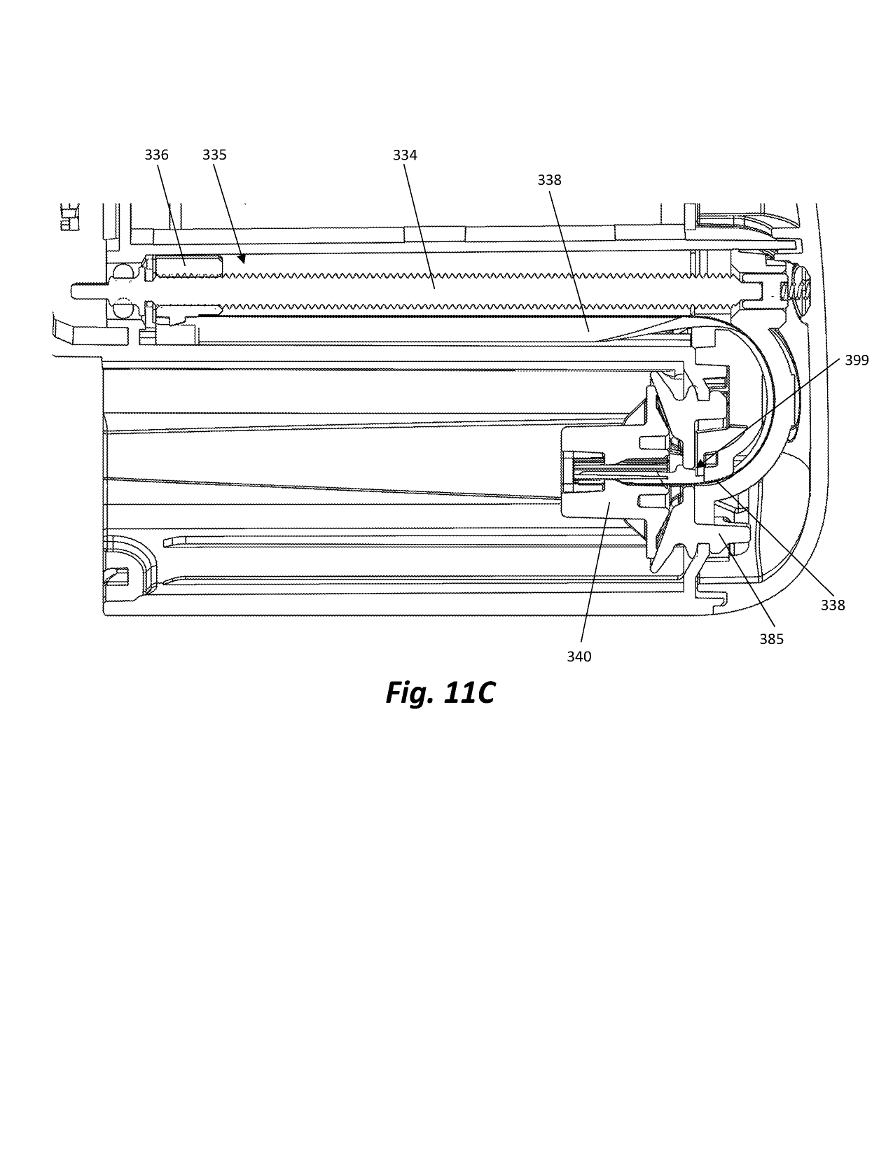

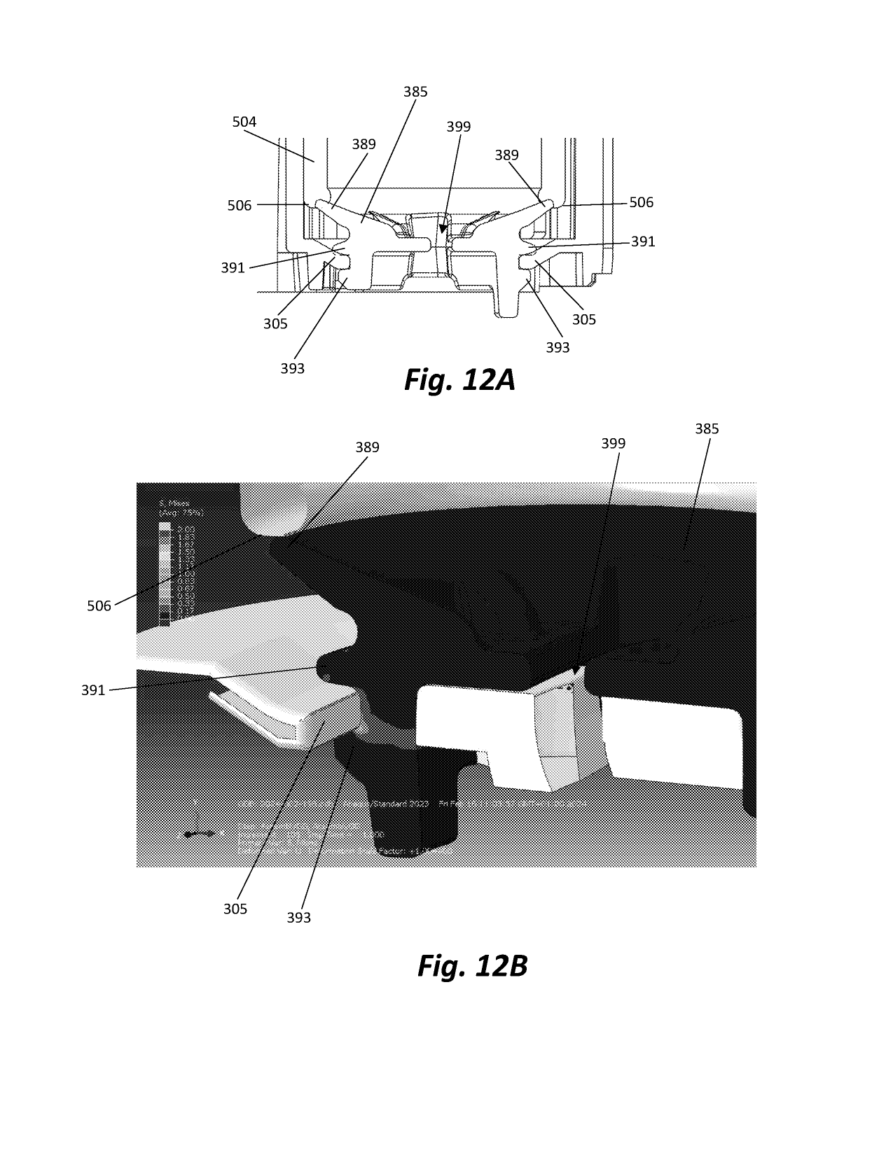

Referring now to FIGS. 11A-1C an exploded view and partial cross-section views depicting an exemplary patch pump system including sealing element 385 are presented. FIGS. 12A-12B depict isolated partial views of the seal created by sealing element. Sealing element 385 is pre-positioned within pump housing 302 at an end of a cartridge recess 303 in pump housing 302 configured to receive the medicament cartridge. Sealing element 385 is configured to be inserted into and interface with a proximal end 504 of cartridge 500 to provide a first seal. In particular, flange 389 in sealing element provides a seal against an edge rim 506 extending around an end of the cartridge 500 at the proximal end 504 of the cartridge. A second seal is provided by a pair of ribs 305, or alternatively, an annular rim, at an end of cartridge recess 303 in pump housing 302 being captured in sealing recesses 395 between annular rim 391 and sealing projections 393. FIG. 11C depicts a patch pump system with a sealing element 385 with the medicament cartridge removed 500 for sake of clarity to depict the interaction of sealing element 385 with pushing system 335. In particular, curved rod 338 of pushing system 335 extends through a central opening 399 in sealing element 385 in order to connect the pushing system 335 to the pusher 340. Curved rod 338 is movable through central opening 399 in order to advance pusher 340 within cartridge 500 to cause medicament to be delivered in response to actuation of pump motor (and also to rewind the pushing system once the cartridge is empty). The seal between cartridge 500 and flange 389 prevents any fluid from leaking through this opening when a cartridge 500 is installed.

It has been found that use of such a sealing element 385 can significantly reduce or eliminate fluid (i.e., insulin from cartridge 500, or, more typically, water when a user takes a shower or otherwise interacts with water while wearing the pump) from escaping the unprotected wet zone area 382 into the protected wet zone area 380 housing the components of the pushing system 335. This prevents such fluid from inhibiting operation of the pushing system 335 and, in particular screw 334, enhancing the ability of the system to provide precise microdoses of insulin to meet the specific needs of the patient.

### Enhanced Rewinding

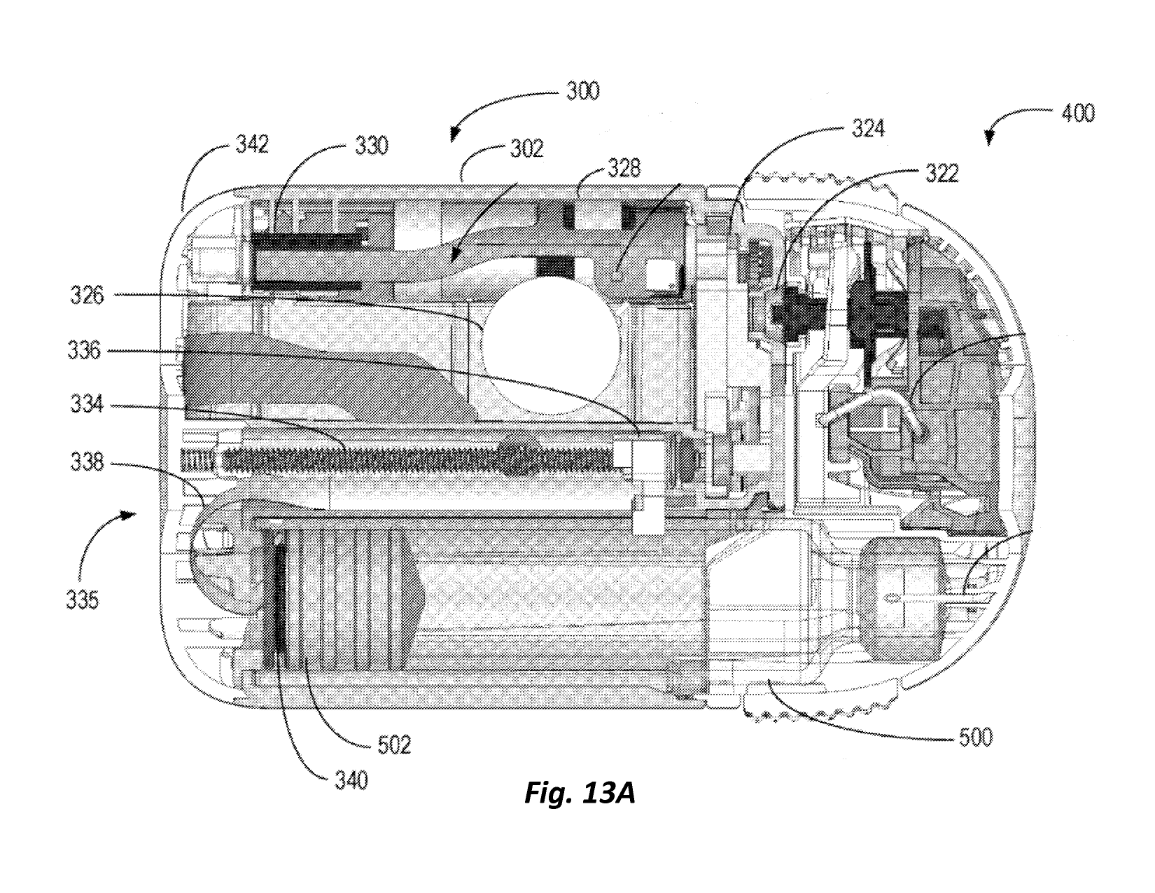

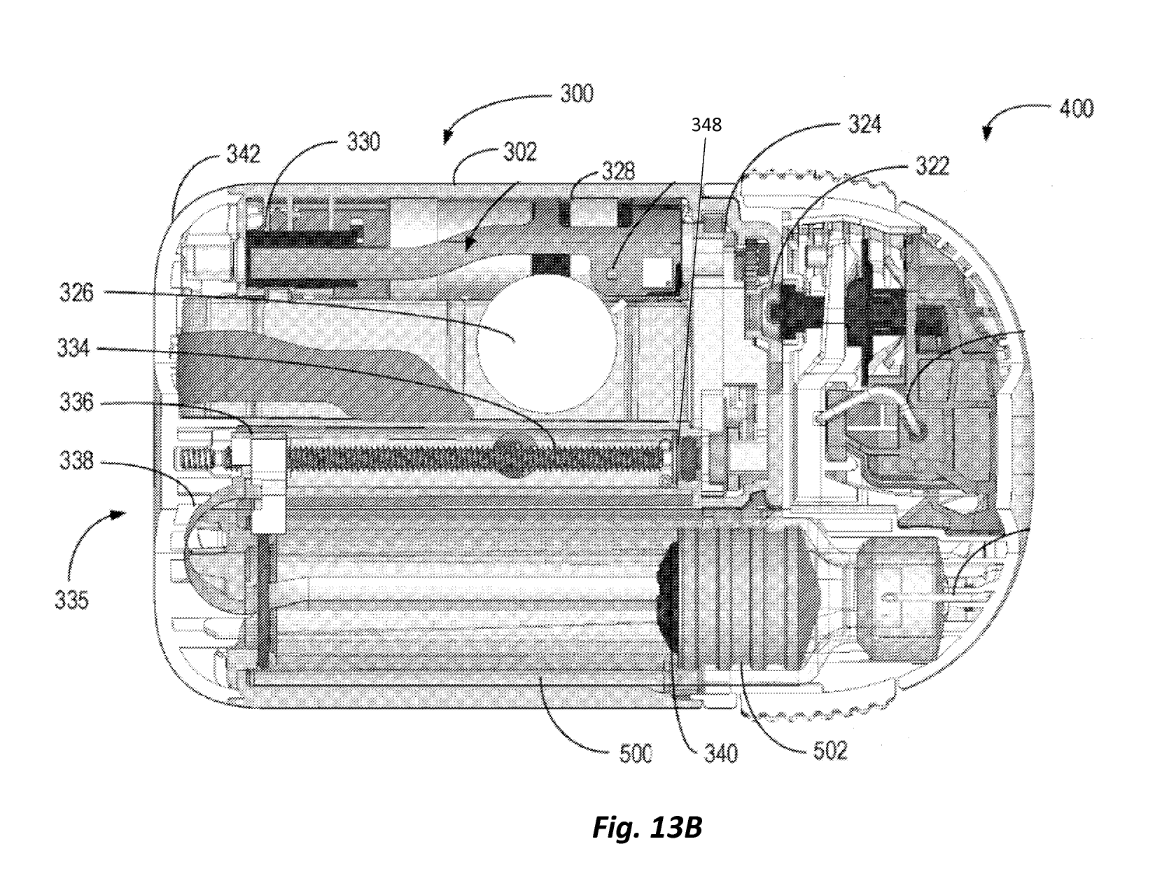

Referring to FIGS. 13A-13B, an exemplary patch pump is shown with the pushing mechanism with a full cartridge (FIG. 13A) and an empty cartridge (FIG. 13B) of medicament. In FIG. 13A, pushing mechanism 335 is depicted in a home position, when cartridge 500 is full of medication. In the home position, nut 336 may be disposed adjacent to the location where screw 334 is coupled to the gearbox 324. In FIG. 13B, pushing mechanism 335 is depicted in a delivery position, after cartridge 500 has delivered medication. Preferably, after all or substantially all of the medication within cartridge 500 has been delivered, the battery of pump 300 should be recharged. Upon reaching a predetermined state in the charging cycle, pushing mechanism 335 is configured to return to the home position. For example, the controller, upon sensing that the battery has reached the predetermined state (e.g., a predetermined time before full charge, such as 20 minutes), will cause pump motor 328 to rewind thereby transitioning the pump back from the empty position of FIG. 13B to the full position of FIG. 13A. In some embodiments, the pump and the cap will not unlock until the battery is sufficiently charged and the pump is in the home position. Gearbox 324 preferably rotates screw 334 in the second direction such that pusher 340 moves away from plunger 502. Nut 336 may continue moving along screw 334 towards gearbox 324 until it reaches a contact sensor 348. Upon sensed contact, the pump firmware is configured to reverse directions of the screw 334 to rotate in the first direction such that nut 336 is advanced a short distance away from gearbox 324 and a small space is created between nut 336 and the contact sensor 348. Resetting pushing mechanism 335 to the home position permits a new, pre-filled cartridge 500 to be inserted into the patch pump.

However, it has been found that during the rewinding phase the screw 334 can become displaced longitudinally within the pump housing 302. In particular, as the nut 336 is rewound along the screw 334 back towards the home position adjacent the gearbox 324 (from the position shown in FIG. 513B to the position shown in FIG. 13A) a longitudinal force is placed on the screw 334 in a direction towards the back housing 342 due to the pusher 340 and cartridge plunger 502 enacting a pulling force on the curved rod 340 in that direction as they are rewound back to the proximal end of cartridge 500. This force can cause the screw 334 to displace longitudinally towards the back housing 342 which creates a collision between the nut 336 and the end of the screw 336 that can affect pump operation.

To address these issues, disclosed herein are systems that employ a spring to apply a preloaded force onto the screw 334. Spring pushes against screw 334 during rewinding to prevent longitudinal displacement within the housing 302 during rewinding (and pumping). Spring also provides an additional benefit regarding measuring of force during rewinding, as will be described in more detail below.

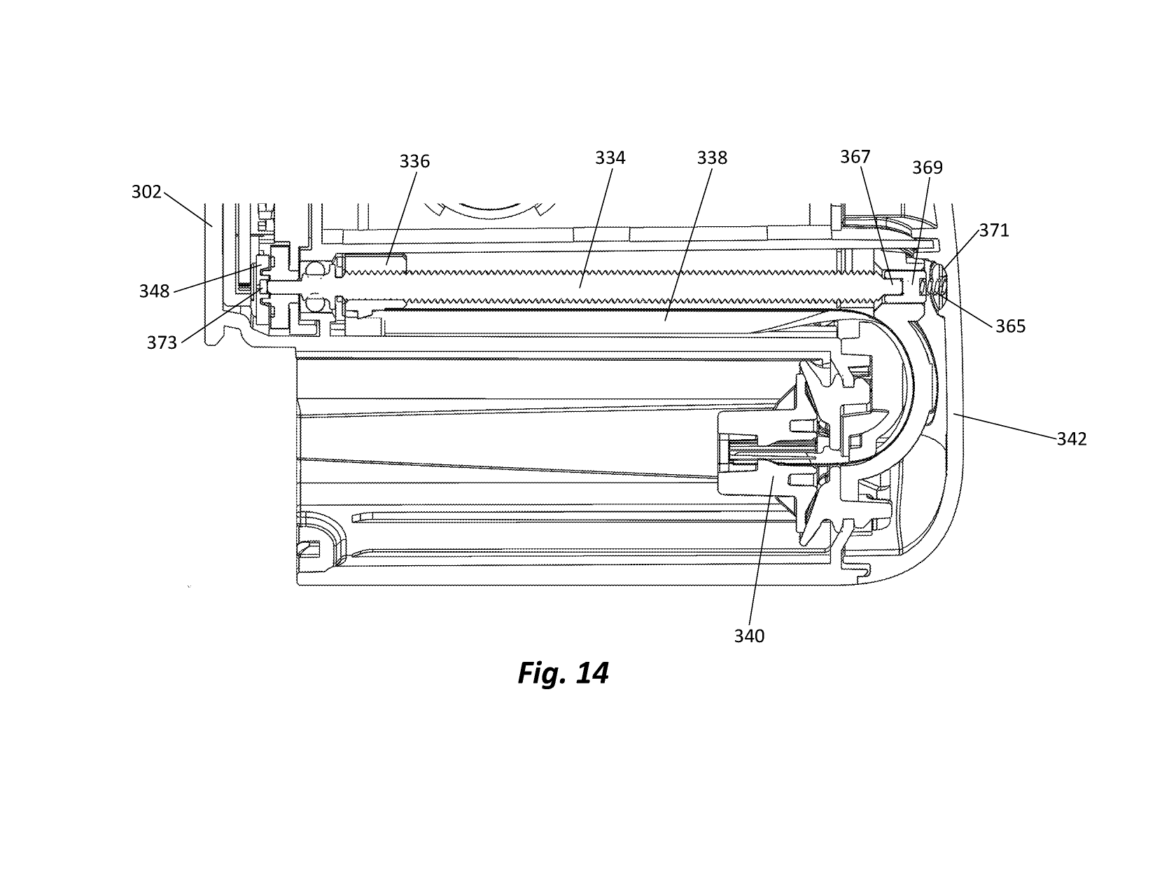

Referring now to FIG. 14, a portion of a patch pump system including a spring 365 that provides a preloaded force onto screw 334 is depicted. In this Figure, nut 336 is depicted in the home position at a proximal end of screw 334 adjacent gearbox when the cartridge is full of medicament. As noted above, once the nut 336 reaches the distal end of the screw 334 (on the right side of FIG. 14), the cartridge is empty and the pushing system 335 must be rewound to bring the nut 336 back to the home position depicted in FIG. 14. Spring 365 effectively functions to prevent the longitudinal displacement of screw 334 during this rewinding phase that can occur as described above.

Screw 334 includes a distal tip 367 that is disposed within a screw bearing 369. Spring 365 is affixed to an opposite side of screw bearing 369 and positioned between screw bearing 369 and an inner surface 371 of back housing 342 such that spring provides a preloaded force onto screw 334 in a direction away from the back housing 342. This force on screw 334 provided by spring 365 counteracts the forces imparted on screw 334 towards the back housing 342 during rewinding to prevent the screw 334 from longitudinally displacing towards the back housing 342. As such, pump rewinding can be accomplished without potentially affecting subsequent operations of the pumping mechanism.

Patch pump system in FIG. 14 also includes a force sensor 348 adjacent an opposite end of screw 334 from the location of the spring 365. As noted above, when spring 365 is not present, during rewinding the only force on the screw 334 is in the direction of back housing 342. As such, the force of the screw 334 in the direction of the force sensor 348 would technically be negative and would not be measured by the force sensor 348 that needs a positive contract force between the screw 334 and the screw contact 373 of force sensor 348. However, because the spring 365 provides a preloaded force onto screw 334, the force sensor 348 can measure the amount of that force (e.g., 2N). Therefore, when rewinding occurs the existing force measurement is reduced by the negative force, but remains a positive, measurable value (e.g., between 0N and 2N). The system can therefore monitor this measured force during rewinding, with any unexpected readings indicating a potential issue with the system, such as, for example, a foreign object within the system blocking the nut 336 and/or the pusher 340 as it is rewound.

### Enhanced Device Reset

Sensor 348 may sense information indicative of the position of the pushing system 335. Sensor 348 preferably is electrically coupled to controller 360 such that the sensed position is sent to controller 360 for processing and detecting the position of the pushing system 335. For example, sensor 348 may indicate that a component of the pushing system 335 is at the end-of-stroke. Sensor 348 may be located adjacent to the pushing system. In a preferred embodiment, sensor 348 is an electrical contact sensor that senses the position of the pushing system 335 based on whether a component (e.g., nut 336) of the pushing system has contacted the sensor.

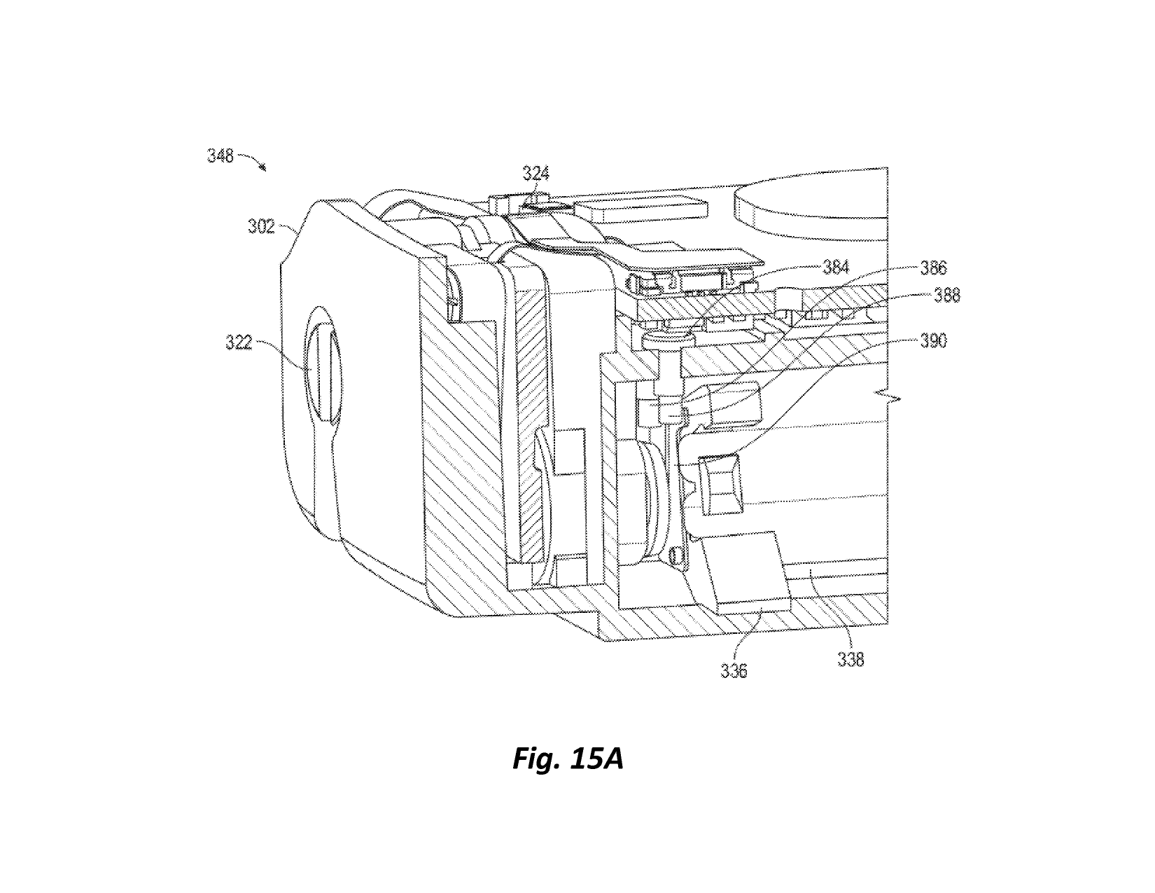

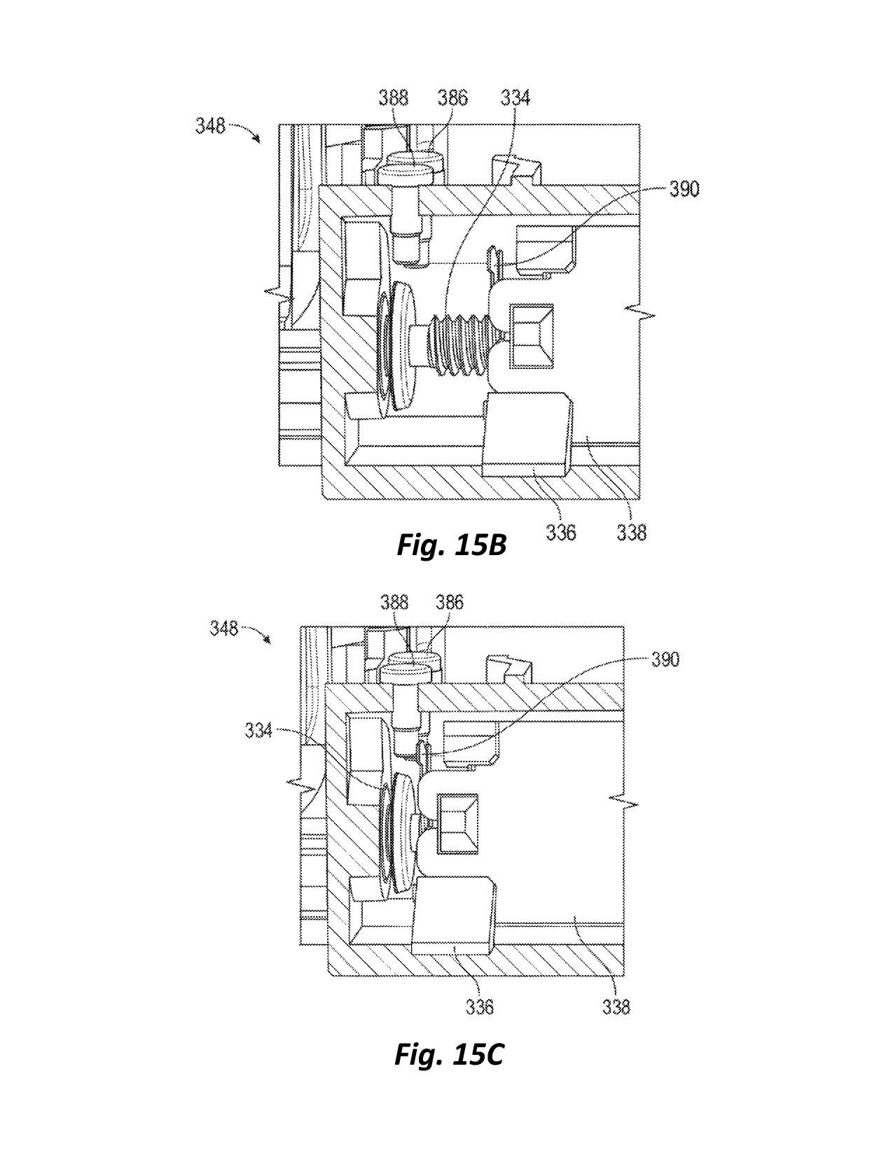

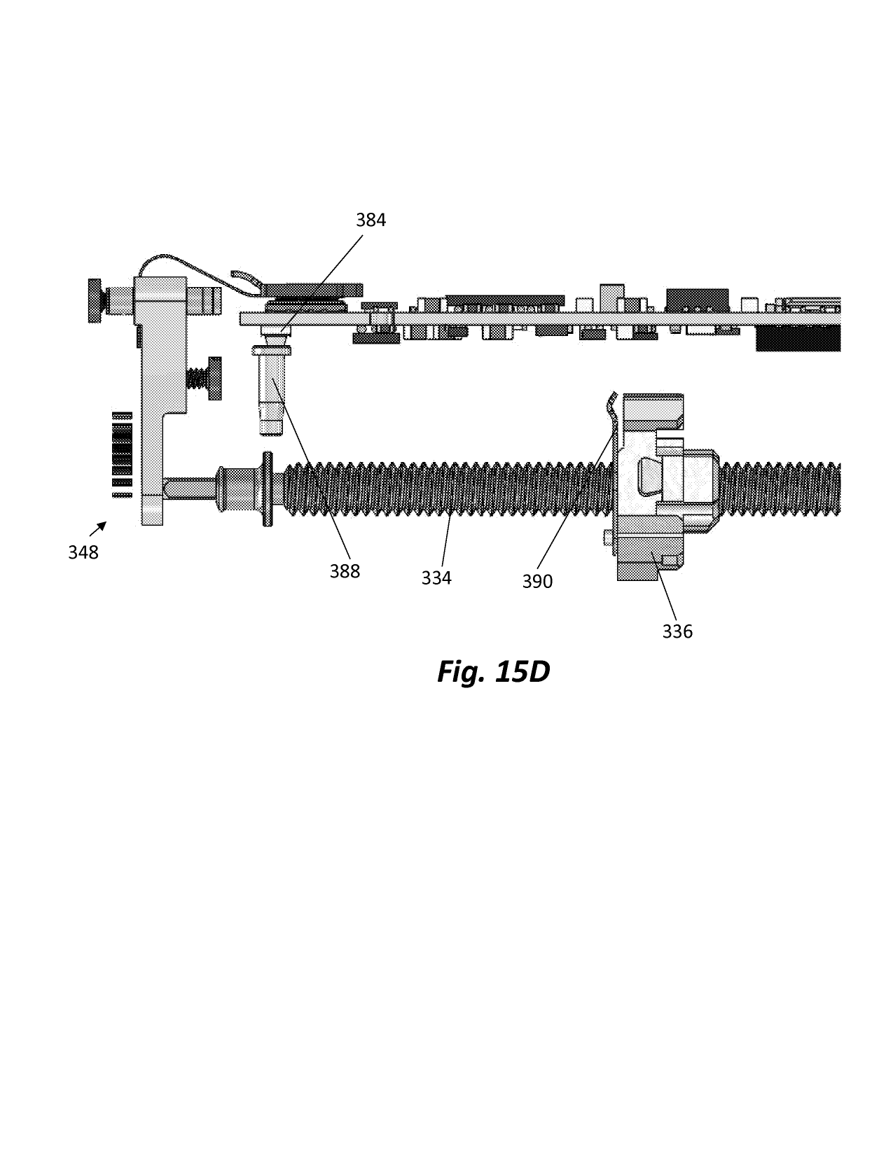

In FIGS. 15A-15D, exemplary contact sensor 348, which may be an electrical contact sensor, is disposed within the pump and configured to detect the position of the pushing system. For example, sensor 348 may include contacting pins 386, 388 that are configured to contact one or more contacting blades 390 coupled to the pushing system. The contacting pins 386, 388 may be coupled to the circuit board via a conductor 384 (which may be a contact spring) such that when the contacting pins 386, 388 contact the one or more contacting blades 390, a circuit an electrical circuit between the pins is completed, which indicates that the pushing system is disposed in a starting position.

The pushing system may include screw 334, nut 336 configured to move along screw 334 such that bendable rod 338, which is coupled to nut 336 pushes a cartridge contactor (not shown) to contact a plunger disposed within the cartridge. Upon movement of the pushing system, the plunger is configured to advance into the cartridge such that medication is moved towards the inflow needle of the cap. After the cartridge is empty, the pushing system moves in an opposite direction, away from the cartridge plunger and back to the starting position. That starting position is rewound and transitions to a first position such that the cartridge is permitted to be removed and exchanged for a subsequent cartridge.

One or more contacting blades 390 may be coupled to nut 336. Sensor 348 preferably is disposed within pump housing 302 and adjacent to contacting blades 390 of pushing system. For example, sensor 348 may include one or more contracting pins 386 and 388 that are configured to contact contacting blade 390 when the pushing system is positioned near the pump housing back such that a new, pre-filled cartridge may be inserted into the patch pump. Contacting pins 386 and 388 may be coupled to conductor 384, which is configured to electrically connect contacting pins 386 and 388 to the circuit board. In FIG. 15B, the contact sensor in a non-contacting position, wherein contacting blades 390 are not connected to contacting pins 386 and 388. In FIG. 15C, the contact sensor in a contacting position, wherein contacting blades 390 are connected to contacting pins 386 and 388.

Sensor 348 may be electrically coupled to a controller such that sensed signals are sent to the controller for processing and determining a state of the patch pump. For example, if sensor 348 detects that the blade (i.e. the nut 336 with the pushing system 335) is in contact with contacting pins 386 and 388, the controller may be configured to cause the pushing mechanism to stop because it reaches the home position (end of stroke).

It has been found that when the contacting blade 390 of the pushing system contacts the contacting pins 386, 388 of the sensor 348 to complete the circuit to indicate that the pushing system is in the home position, an electrical current is established that can lead to corrosion at the contact points between the contacting pins 386, 388 and the contacting blades 390. This is due to the charge in the circuit not being balanced such that a positive voltage is always applied to the same side and the current is going in the same direction (i.e., one pin always positive and one pin always negative). This is possible by having the polarity always applied to the same blade or contacting pin and when an electrolyte in the chamber like insulin or salty water allowing the current to circulate. The blade or contacting pin acts as anode or cathode depending on the polarity. The anode is the metal where oxidation occurs. The cathode is the metal where reduction occurs.

FIG. 17 shows the effect of corrosion on the anode (circled reasons). This corrosion is influenced by electrode material, potential difference between anode and cathode and the time duration at which the electrodes are exposed to the circulation of current.

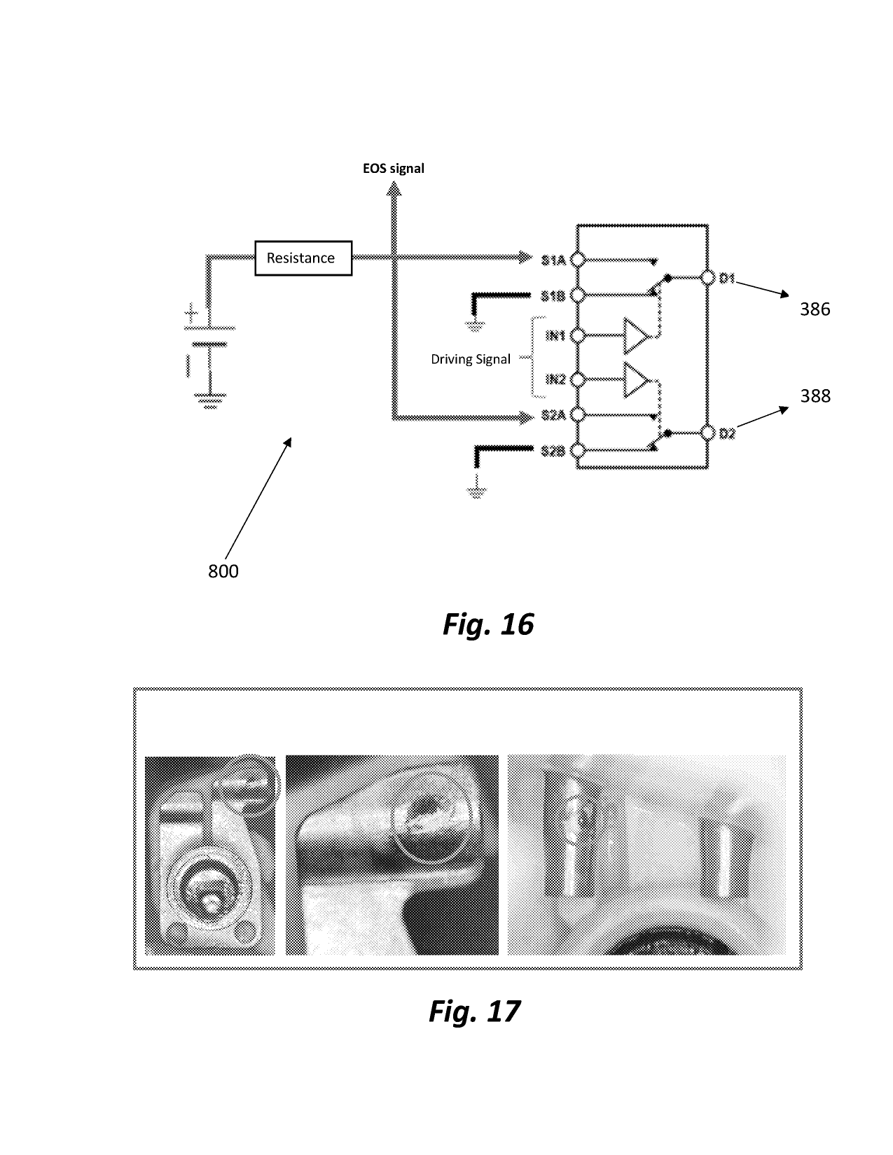

As such, disclosed herein and depicted in FIG. 16 is a redesigned end of stroke circuit 800 with charge balancing that addresses this issue. This end of stroke circuit is formed when the contacting blade 390 contacts the contacting pins 386, 388 to electrically signal to the processor that the nut 336 has been fully rewound along the screw 334 (i.e., “end of stroke). In circuit 800, the voltage is frequently reversed such that current is going in both directions. In other words, the new circuit alternately polarizes the pins 386, 388 so that the current flows in both directions (each time the pins are reversed: this removes the charges that had built up in the electrolyte). The enables a grounding phase that discharges any remaining accumulated charge when the contacting blade 390 contacts the contacting pins 386, 388. This eliminates the accumulation of charges at the contacting point between the blade 390 and the pins 386, 388 noted above, and accordingly the corrosion that can result from this repeated discharge each time the system is rewound.

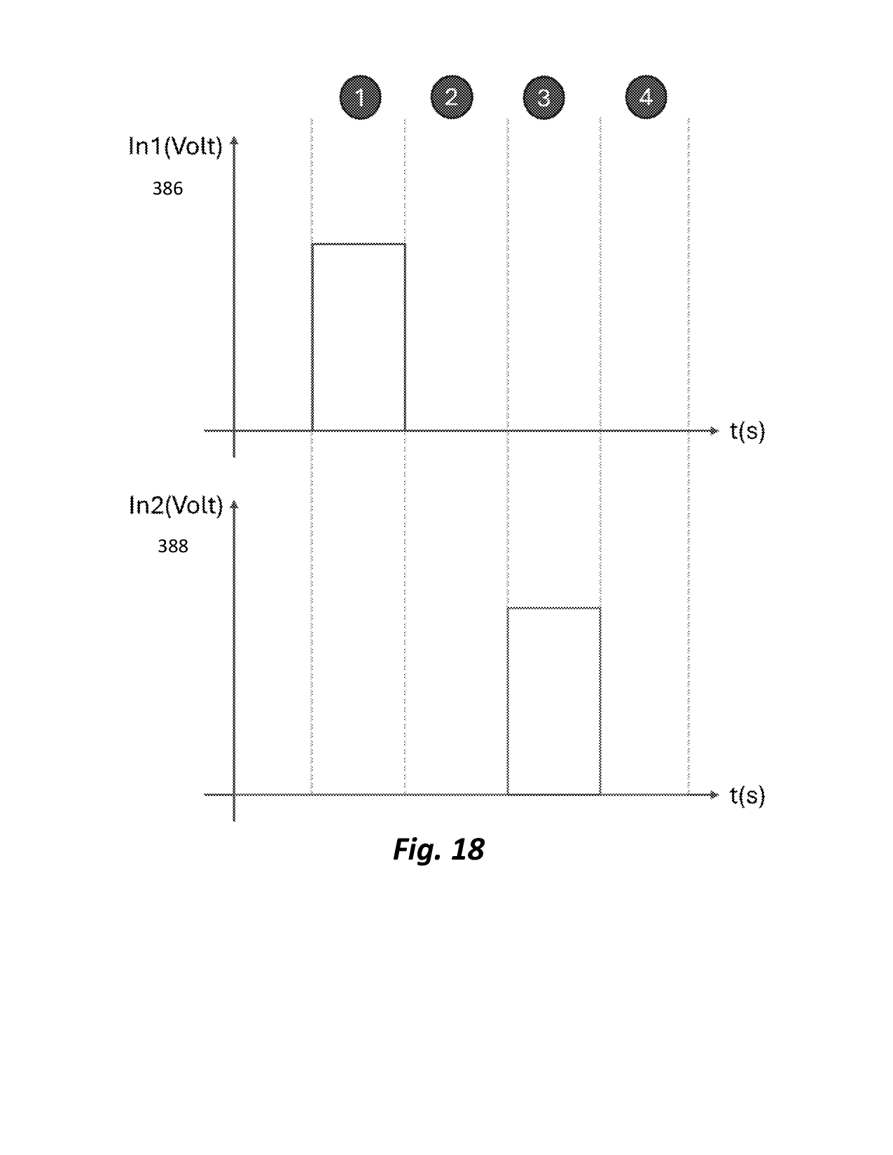

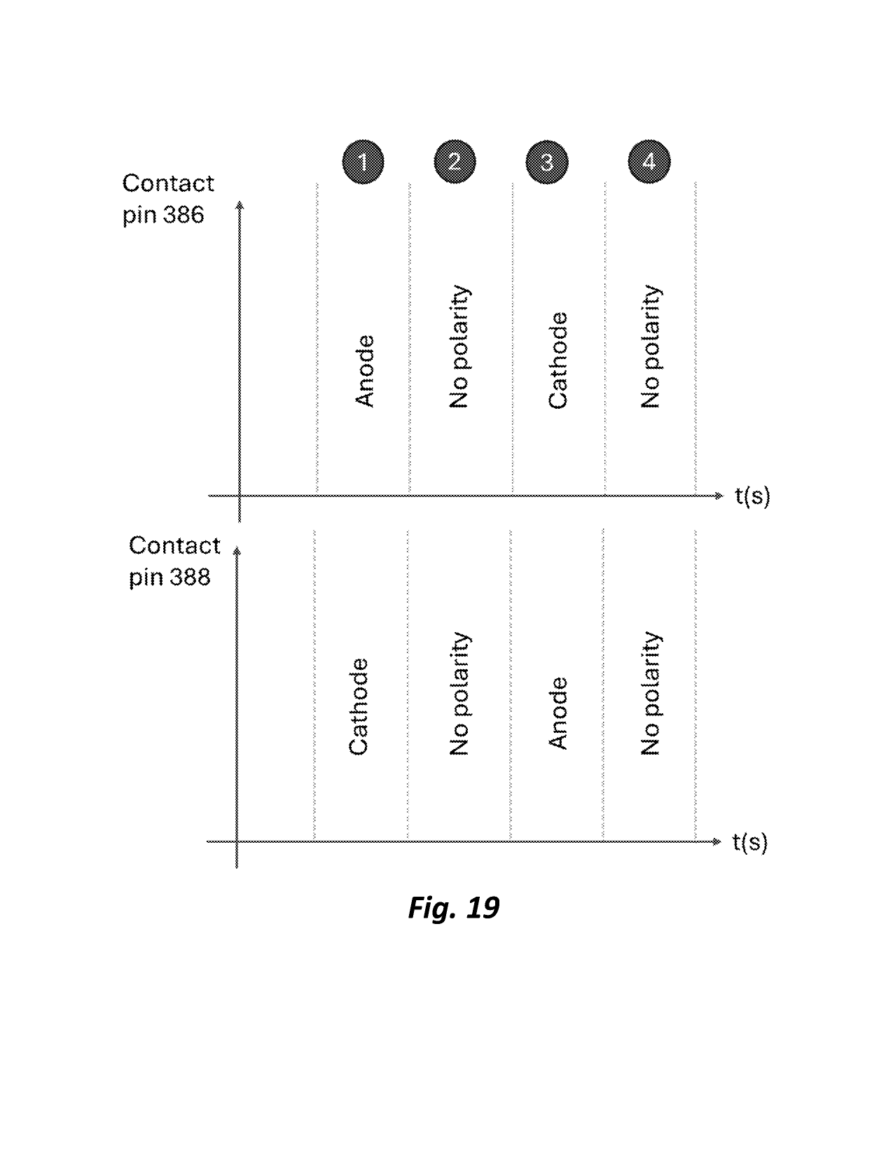

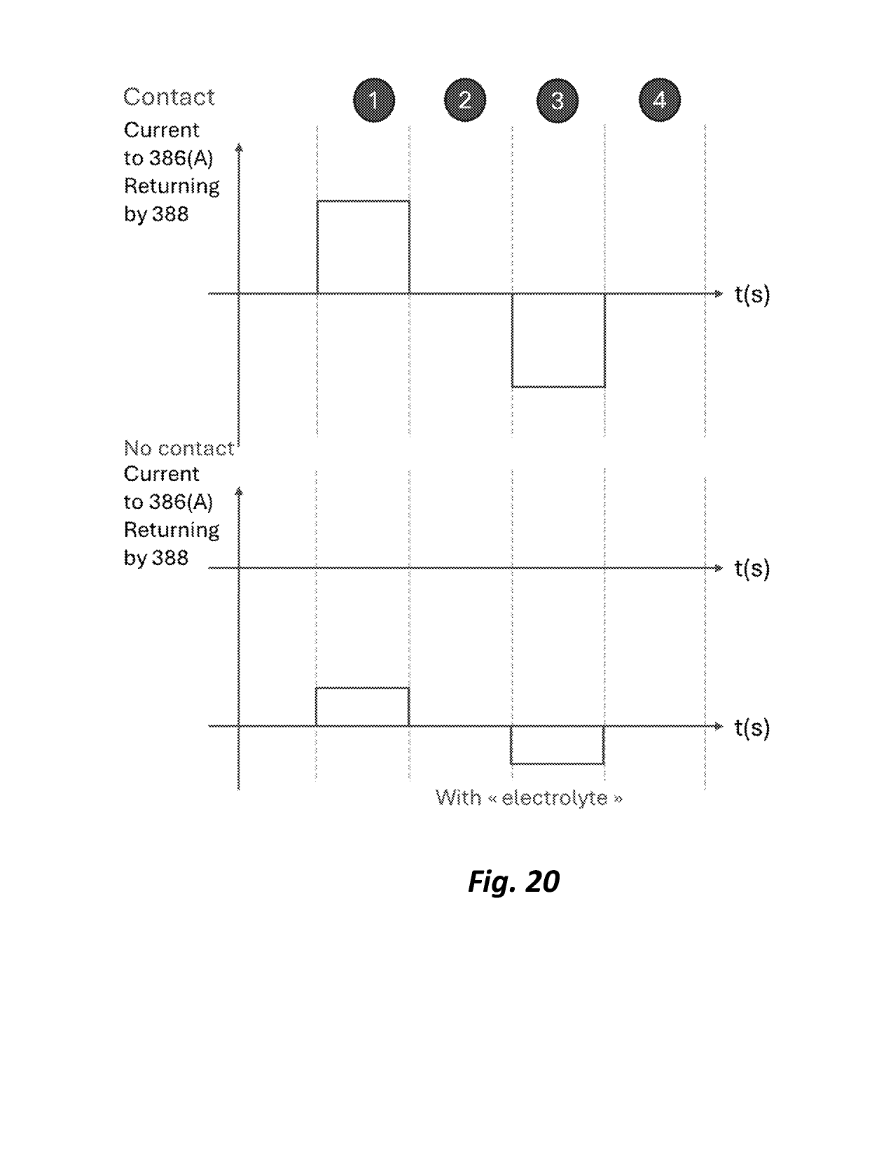

In circuit 800, the voltage is frequently reversed such that each contact pin 386, 388 or blade is acting as anode and cathode at times during the period divided in 4 phases as depicted in FIG. 19. FIG. 18 describes the sequence and the driving signal of the switch. The contacting pins 386, 388 are connected to S1A/S2A when the driving signal IN1/IN2 is high and connected to S1B/S2B when the driving signal IN1/IN2 is low. In the phase 1 contacting pin 386 is acting as an anode while the pin 388 as a cathode while in the phase 3 the contacting pin 386 is acting as a cathode while the 388 as an anode. In the phase 2 and 4 the contacting pins or blade are grounded and connected together for discharging the remaining charge accumulated in the electrolyte. This result is a charge balancing where each contacting pin acts as an anode in a limited time and where the current circulates in both directions as indicated in FIG. 20

A key element of this end of stroke detection design is that it functions properly not only in dry conditions but also in wet conditions. As noted above, although the previous electrical latch principle powered with DC current works well for a basic end of stroke detection based on a moving latch (390) shorting two pins(386, 388), the drawback of that principle is when the environment becomes wet (ex: the cavity gets filled with water or worse, with salty water) there can be corrosion effects that will damage the metallic contacts over time (even with stainless steels, titanium, etc...) as depicted in FIG. 17. To get rid of that corrosion phenomenon, the latch is powered with alternate voltage at high frequency in order to balance the oxidation/ reduction phenomenon at each cycle. This is what is called charge balancing and avoids any corrosion over time by limiting/reverting the electrochemical reactions at each cycle very accurately. The goal being to neutralize all the injected charges in one direction by the same amount of charge in the other direction (see FIG. 20 for details). This avoids to change the pH of the liquid. The system can use a passive charge balancing in which there is a phase where the 2 pins (386 & 388) are electrically shorted through the circuit to allow any residual charge to be balanced and keep the liquid as neutral as possible. In FIG. 20, the 4th phase of the signal corresponds to that residual charge balancing phase.

In an embodiment, a medication infusion device can include a pump housing including a cartridge recess configured to receive a medication cartridge and a pump motor disposed within the pump housing configured to operate a pushing system to cause a medication in the medication cartridge to be delivered to the user. A sealing element can be disposed within the cartridge recess and be configured to provide a first seal with the medication cartridge and a second seal with the pump housing to prevent fluid from traveling from a wet zone region in the pump housing that includes the cartridge recess into a protected area of the pump housing that includes the pushing system.

In some embodiments, the sealing element includes a flexible flange configured to form the first seal with an annular rim at a proximal end of the medicament cartridge.

In some embodiments, the sealing element forms the second seal by capturing a portion of the pump housing in one or more sealing recesses defined between an annular rim and one or more sealing projections of the sealing element.

In some embodiments, the sealing element comprises a central enabling a portion of the pushing system to pass through the sealing element.

In an embodiment, a medication infusion device can include a patch pump configured to be removably adhered to a wearer’s skin for delivering doses of medication to the wearer through a transcutaneous portion, the patch pump including a pump housing and a pump motor disposed within the pump housing, the pump motor configured to pump the medication towards the transcutaneous portion. A force absorber can be configured to absorb forces within the pump housing.

In some embodiments, the medication infusion device further includes a vibration motor separate from the pump motor disposed within the pump housing and a controller operatively coupled to the pump motor and the vibration motor, the controller configured to cause the vibration motor to vibrate to provide alerts to the wearer. The force absorber can be a vibration holder partially surrounding a portion of the vibration motor, the vibration holder configured to absorb the vibrations of the vibration motor to transmit the vibrations to the wearer external of the pump housing while preventing the vibrations from affecting other components internal to the pump housing.

In some embodiments, the vibration motor comprises a DC motor portion and an eccentric rotating mass, and the vibration holder is positioned on the DC motor portion.

In some embodiments, an inner surface of vibration holder conforms to a shape of DC motor portion.

In some embodiments, an outer surface of vibration holder confirms to a shape of an inner surface of the pump housing.

In some embodiments, the vibration holder comprises a single monolithic piece of material.

In some embodiments, the medication infusion device further includes a plunger and a pusher connecting the pump motor to the plunger such that operation of the pump motor causes the pusher to move the plunger to deliver the medication to the wearer, the pusher including a curved rod. The force absorber can be a bender disposed between the curved rod and the housing to absorb forces imparted by the curved rod during pumping operations that would otherwise be imparted to the pump housing to prevent a pump housing back from separating from a main pump housing of the pump housing due at least in part to those forces.

In some embodiments, the bender comprises a curved inner surface conforming to a shape of the curved rod.

In some embodiments, the bender disposed between the curved rod and the housing is an outer bender, and further comprising an inner bender disposed on an inside of the curved rod.

In some embodiments, the inner bender comprises a curved outer surface conforming to a shape of the curved rod.

In some embodiments, the outer bender and the inner bender each comprise a single, monolithic piece of material.

In an embodiment, medication infusion device can include a patch pump configured to be removably adhered to a wearer’s skin for delivering doses of medication from a container through a transcutaneous portion. The patch pump can include a pushing mechanism configured to be coupled to a plunger of the container, the pushing mechanism including a screw and a nut configured to move along the screw in a first direction to advance a pusher to move the plunger. A pump motor can be coupled to the pushing mechanism and configured to move the pusher towards the plunger of the container by advancing the nut along the screw in the first direction such that medication is moved out of the container. The pump motor can be further configured to rewind the nut along the screw in a second direction opposite of the first direction when the container is empty of medication.

In some embodiments, the medication infusion device can include a spring providing a preloaded force onto the screw that counteracts forces imparted onto the screw by the pushing mechanism when the nut is rewound along the screw in the second direction.

In some embodiments, the spring is disposed between a screw bearing attached to a distal tip of the screw and an inner surface of the pump housing.

In some embodiments, the medication infusion device can further include one or more contacting pins and a contacting blade affixed to the pushing mechanism. The contracting blade can be configured to contact the one or more contacting pins when the pushing mechanism is fully rewound to send an electrical signal to a processor that the pushing mechanism is fully rewound. An electrical circuit formed when the contacting blade contacts the one or more contacting pins can be configured to discharge any remaining accumulated charge.

In some embodiments, discharging the remaining accumulated charge prevents corrosion from developing where the contacting blade contacts the one or more contacting pins.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to producenumerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

1. A medication infusion device, comprising:
a pump housing including a cartridge recess configured to receive a medication cartridge;
a pump motor disposed within the pump housing configured to operate a pushing system to cause a medication in the medication cartridge to be delivered to the user; and
a sealing element disposed within the cartridge recess and configured to provide a first seal with the medication cartridge and a second seal with the pump housing to prevent fluid from traveling from a wet zone region in the pump housing that includes the cartridge recess into a protected area of the pump housing that includes the pushing system.

2. The medication infusion device of claim 1, wherein the sealing element includes a flexible flange configured to form the first seal with an annular rim at a proximal end of the medicament cartridge.

3. The medication infusion device of claim 1, wherein the sealing element forms the second seal by capturing a portion of the pump housing in one or more sealing recesses defined between an annular rim and one or more sealing projections of the sealing element.

4. The medication infusion device of claim 1, wherein the sealing element comprises a central enabling a portion of the pushing system to pass through the sealing element.

5. A medication infusion device, comprising:
a patch pump configured to be removably adhered to a wearer’s skin for delivering doses of medication to the wearer through a transcutaneous portion, the patch pump including a pump housing;
a pump motor disposed within the pump housing, the pump motor configured to pump the medication towards the transcutaneous portion; and
a force absorber configured to absorb forces within the pump housing.

6. The medication infusion device of claim 5, further comprising:
a vibration motor separate from the pump motor disposed within the pump housing;
a controller operatively coupled to the pump motor and the vibration motor, the controller configured to cause the vibration motor to vibrate to provide alerts to the wearer; and
wherein the force absorber is a vibration holder partially surrounding a portion of the vibration motor, the vibration holder configured to absorb the vibrations of the vibration motor to transmit the vibrations to the wearer external of the pump housing while preventing the vibrations from affecting other components internal to the pump housing.

7. The medication infusion device of claim 6, wherein the vibration motor comprises a DC motor portion and an eccentric rotating mass, and wherein the vibration holder is positioned on the DC motor portion.

8. The medication infusion device of claim 7, wherein an inner surface of vibration holder conforms to a shape of DC motor portion.

9. The medication infusion device of claim 6, wherein an outer surface of vibration holder confirms to a shape of an inner surface of the pump housing.

10. The medication infusion device of claim 6, wherein the vibration holder comprises a single monolithic piece of material.

11. The medication infusion device of claim 5, further comprising:
a plunger;
a pusher connecting the pump motor to the plunger such that operation of the pump motor causes the pusher to move the plunger to deliver the medication to the wearer, the pusher including a curved rod; and
wherein the force absorber is a bender disposed between the curved rod and the housing to absorb forces imparted by the curved rod during pumping operations that would otherwise be imparted to the pump housing to prevent a pump housing back from separating from a main pump housing of the pump housing due at least in part to those forces.

12. The medication infusion device of claim 11, wherein the bender comprises a curved inner surface conforming to a shape of the curved rod.

13. The medication infusion device of claim 11, wherein the bender disposed between the curved rod and the housing is an outer bender, and further comprising an inner bender disposed on an inside of the curved rod.

14. The medication infusion device of claim 13, wherein the inner bender comprises a curved outer surface conforming to a shape of the curved rod.

15. The medication infusion device of claim 13, wherein the outer bender and the inner bender each comprise a single, monolithic piece of material.

16. A medication infusion device comprising a patch pump configured to be removably adhered to a wearer’s skin for delivering doses of medication from a container through a transcutaneous portion, the patch pump comprising:
a pushing mechanism configured to be coupled to a plunger of the container, the pushing mechanism including a screw and a nut configured to move along the screw in a first direction to advance a pusher to move the plunger; and
a pump motor coupled to the pushing mechanism and configured to move the pusher towards the plunger of the container by advancing the nut along the screw in the first direction such that medication is moved out of the container, wherein the pump motor is further configured to rewind the nut along the screw in a second direction opposite of the first direction when the container is empty of medication.

17. The medication infusion device of claim 16, further comprising a spring providing a preloaded force onto the screw that counteracts forces imparted onto the screw by the pushing mechanism when the nut is rewound along the screw in the second direction.

18. The medication infusion device of claim 17, wherein the spring is disposed between a screw bearing attached to a distal tip of the screw and an inner surface of the pump housing.

19. The medication infusion device of claim 16, further comprising:
one or more contacting pins; and
a contacting blade affixed to the pushing mechanism, wherein the contracting blade is configured to contact the one or more contacting pins when the pushing mechanism is fully rewound to send an electrical signal to a processor that the pushing mechanism is fully rewound, wherein an electrical circuit formed when the contacting blade contacts the one or more contacting pins is configured to discharge any remaining accumulated charge.

20. The medication infusion device of claim 19, wherein discharging the remaining accumulated charge prevents corrosion from developing where the contacting blade contacts the one or more contacting pins.
